(19) United States
(12) Patent Application Publication
(10) Pub. No.: US 2026/0102248 A1
(43) Pub. Date: Apr. 16, 2026

(54) BALLOON CATHETERS FOR PROSTHETIC IMPLANTS

(71) Applicant: Edwards Lifesciences Corporation, Irvine, CA (US)

(72) Inventors: Grant Jason Gangeness, Carlsbad, CA (US); Tri D. Tran, Fountain Valley, CA (US); Yidong M. Zhu, Tustin, CA (US); Sean Chow, Irvine, CA (US); Ronaldo C. Cayabyab, Mission Viejo, CA (US)

(21) Appl. No.: 19/415,776

(22) Filed: Dec. 11, 2025

Related U.S. Application Data
(63) Continuation of application No. PCT/US2024/033382, filed on Jun. 11, 2024.
(60) Provisional application No. 63/507,713, filed on Jun. 12, 2023.

(51) Int. Cl. A61F 2/24

(52) U.S. Cl. CPC A61F 2/2433; A61F 2/2436; A61F 2230/0067

(57) ABSTRACT

A delivery apparatus for a prosthetic heart valve can comprise a shaft having a proximal end portion and a distal end portion, and an inflatable balloon mounted on the distal end portion of the shaft. The inflatable balloon can comprise a first conical portion, a second conical portion axially disposed relative to the first conical portion, and a central longitudinal axis extending between the first conical portion and the second conical portion. The first conical portion can have a first axial length and the second conical portion can have a second axial length that is different than the first axial length. The first conical portion has a first slope relative to the central longitudinal axis and the second conical portion has a second slope relative to the central longitudinal axis that is different than the first slope.

### CROSS REFERENCE TO RELATED APPLICATION

This application is a continuation of PCT patent application no. PCT/2024/033382 filed on Jun. 11, 2024, which claims the benefit of U.S. Provisional Patent Application No. 63/507,713, filed Jun. 12, 2023, each of these applications being incorporated by reference herein in its entirety.

### FIELD

The present disclosure relates to balloon catheters for prosthetic implants.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (for example, through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a sheath of the delivery apparatus so that the prosthetic heart valve can self-expand to its functional size.

In some examples of an implantation procedure, at least some portions of a prosthetic implant (such as a prosthetic heart valve) may be insufficiently expanded such that the prosthetic implant is not expanded to its functional size. Accordingly, a need exists for ensuring that the prosthetic implant is sufficiently expanded to its functional size.

### SUMMARY

Described herein are balloon catheters and methods for implanting prosthetic heart valves. The disclosed devices and methods can, for example, provide for improved expansion of prosthetic implants. As such, the devices and methods disclosed herein can, among other things, overcome one or more of the deficiencies of typical prosthetic heart valves and their delivery apparatus.

The foregoing and other objects, features, and advantages of the disclosed technology will become more apparent from the following detailed description, which proceeds with reference to the accompanying figures.

A delivery apparatus for a prosthetic heart valve can comprise a shaft having a distal end portion and an inflatable balloon mounted on the distal end portion of the shaft.

In some examples, the balloon can comprise a first conical portion and a second conical portion axially disposed relative to the first conical portion.

In some examples, a central longitudinal axis can extend between the first conical portion and the second conical portion.

In some examples, the first conical portion can have a first axial length and the second conical portion can have a second axial length different than the first axial length.

In some examples, the first axial length can be greater than the second axial length.

In some examples, the first conical portion can have a first slope relative to the central longitudinal axis and the second conical portion has a second slope relative to the central longitudinal axis.

In some examples, the second slope can be different than the first slope.

In some examples, the second slope can be greater than the first slope.

In some examples, the inflatable balloon can further comprise a cylindrical intermediate portion disposed between the first conical portion and the second conical portion.

In some examples, the cylindrical intermediate portion can have an axial length, and the axial length of the cylindrical intermediate portion can be greater than the second axial length of the second conical portion.

In one representative example, a delivery apparatus for a prosthetic heart valve can comprise a shaft having a proximal end portion and a distal end portion and an inflatable balloon mounted on the distal end portion of the shaft. The balloon can comprise a first conical portion having a first axial length, a second conical portion axially disposed relative to the first conical portion, and a central longitudinal axis extending between the first conical portion and the second conical portion. The second conical portion can have a second axial length different than the first axial length. The first conical portion can have a first slope relative to the central longitudinal axis, and the second conical portion can have a second slope relative to the central longitudinal axis that is different than the first slope.

In one representative example, a system can comprise a docking device, a prosthetic heart valve comprising a generally cylindrical stent frame with an axial opening, and a balloon configured to extend through the axial opening of the stent frame. The stent frame of the prosthetic heart valve can further comprise a proximal end portion, a distal end portion, and an intermediate portion between the proximal end portion and the distal end portion. The prosthetic heart valve can be configured to be positioned within the docking device. The balloon can comprise a cylindrical intermediate portion, a proximal frustoconical portion tapering at a first slope relative to a central longitudinal axis of the balloon in a proximal direction from the cylindrical intermediate portion, and a distal frustoconical portion tapering at a second slope relative to the central longitudinal axis of the balloon in a distal direction from the cylindrical intermediate portion. The intermediate portion, the proximal frustoconical portion, and the distal frustoconical portion can be disposed along the central longitudinal axis. The balloon, in an inflated state, can be configured to exert a first pressure against the intermediate portion of the stent frame. The balloon, in the inflated state, is configured to expand and exert a second, lesser pressure against one of the proximal end portion and the distal end portion of the stent frame.

In one representative example, a balloon can comprise a central longitudinal axis, a first neck portion, a first conical portion tapering to the first neck portion at a first slope relative to the central longitudinal axis and in a first direction, the first conical portion having a first axial length, a second conical portion tapering to the first conical portion at a second slope relative to the central longitudinal axis and in the first direction, the second conical portion having a second axial length, an intermediate portion axially adjacent the second conical portion, the intermediate portion having an axial length and a radial diameter, a third conical portion tapering from the intermediate portion at a third slope relative to the central longitudinal axis and in a second direction, the third conical portion having a third axial length, a fourth conical portion tapering from the intermediate portion at a fourth slope relative to the central longitudinal axis and in the second direction, the fourth conical portion having a fourth axial length; and a second neck portion.

In one representative example, a balloon can comprise a central longitudinal axis, a first frustoconical portion disposed on the central longitudinal axis, the first frustoconical portion having a first axial length and a first slope relative to the central longitudinal axis, a second frustoconical portion axially adjacent the first frustoconical portion and tapering to the first frustoconical portion, the second frustoconical portion having a second axial length and a second slope relative to the central longitudinal axis, a third frustoconical portion axially adjacent the second frustoconical portion and tapering to the second frustoconical portion, the third frustoconical portion having a third axial length and a third slope relative to the central longitudinal axis, a cylindrical intermediate portion axially adjacent third frustoconical portion, the cylindrical intermediate portion having an axial length and a radial diameter, a fourth frustoconical portion axially adjacent the cylindrical intermediate portion and tapering from the cylindrical portion, the fourth frustoconical portion having a fourth axial length and a fourth slope relative to the central longitudinal axis, a fifth frustoconical portion axially adjacent the fourth frustoconical portion and tapering from the fourth frustoconical portion, the fifth frustoconical portion having a fifth axial length and a fifth slope relative to the central longitudinal axis, and a sixth frustoconical portion axially adjacent the fifth frustoconical portion and tapering from the fifth frustoconical portion, the sixth frustoconical portion having a sixth axial length and a sixth slope relative to the central longitudinal axis.

In one representative example, a method can comprise: implanting a docking device in a native annulus of a subject's heart; using a delivery apparatus, advancing a prosthetic heart valve mounted to an inflatable balloon of the delivery apparatus to the native annulus; positioning the prosthetic heart valve and the inflatable balloon within the docking device; and inflating the balloon such that the balloon exerts a greater pressure on an intermediate portion of the prosthetic heart valve than one of a proximal end and a distal end of the prosthetic heart valve.

In some examples, a delivery apparatus comprises one or more of the components recited in Examples 1-40 below.

The above method(s) can be performed on a living animal or on a simulation, such as on a cadaver, cadaver heart, anthropomorphic ghost, simulator (for example, with body parts, heart, tissue, etc. being simulated).

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 schematically illustrates a stage in an example mitral valve replacement procedure where a guide catheter and a guidewire are inserted into a blood vessel of a patient and navigated through the blood vessel and into a heart of the patient, towards a native mitral valve of the heart.

FIG. 2A schematically illustrates another stage in the example mitral valve replacement procedure where a docking device delivery apparatus extending through the guide catheter is implanting a docking device for a prosthetic heart valve at the native mitral valve.

FIG. 2B schematically illustrates another stage in the example mitral valve replacement procedure where the docking device of FIG. 2A is fully implanted at the native mitral valve of the patient and the docking device delivery apparatus has been removed from the patient.

FIG. 3A schematically illustrates another stage in the example mitral valve replacement procedure where a prosthetic heart valve delivery apparatus extending through the guide catheter is implanting a prosthetic heart valve in the implanted docking device at the native mitral valve.

FIG. 3B schematically illustrates another stage in the example mitral valve replacement procedure where the prosthetic heart valve is fully implanted within the docking device at the native mitral valve and the prosthetic heart valve delivery apparatus has been removed from the patient.

FIG. 4 schematically illustrates another stage in the example mitral valve replacement procedure where the guide catheter and the guidewire have been removed from the patient.

FIG. 5 is side view of an exemplary guide catheter configured to receive a delivery apparatus and guide the delivery apparatus through a portion of a patient's vasculature, according to an example.

FIG. 6 is a side view of a delivery apparatus for a docking device, according to an example.

FIG. 7 is a side view of a delivery apparatus for a docking device, according to an example.

FIG. 8 is a perspective view of a docking device, according to an example.

FIG. 9 is a perspective view of a delivery apparatus for a prosthetic heart valve, according to an example.

FIG. 10 is a side view of a distal end portion of the delivery apparatus of FIG. 9 comprising an inflatable balloon, according to an example.

FIG. 11 is a side view of a distal end portion of the delivery apparatus of FIG. 9 comprising an inflatable balloon, according to an example.

FIG. 12 is a side view of a distal end portion of the delivery apparatus of FIG. 9 comprising an inflatable balloon, according to an example.

FIG. 13 is a side view of a distal end portion of the delivery apparatus of FIG. 9 comprising an inflatable balloon, according to an example.

FIG. 14 is a perspective view of a prosthetic heart valve for use with the delivery apparatus of FIG. 9, according to an example.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises. ” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (for example, out of the patient's body), while distal motion of the device is motion of the device away from the user and toward the implantation site (for example, into the patient's body). The terms “longitudinal” and “axial” refer to an axis extending in the proximal and distal directions, unless otherwise expressly defined.

As used herein, “e.g.” means “for example,” and “i.e.” means “that is.”

### Introduction to the Disclosed Technology

Described herein are examples of a steerable delivery apparatus (sometimes referred to as a steerable catheter) that can be used to navigate a subject's vasculature to deliver an implantable, expandable medical device (such as a docking device or a prosthetic heart valve), tools, agents, or other therapy to a location within the body of a subject. Examples of procedures in which the steerable catheters are useful include neurological, urological, gynecological, fertility (such as in vitro fertilization, artificial insemination), laparoscopic, arthroscopic, transesophageal, transvaginal, transvesical, transrectal, and procedures including access in any body duct or cavity. Particular examples include placing implants, including stents, grafts, embolic coils, and the like; positioning imaging devices and/or components thereof, including ultrasound transducers; and positioning energy sources, for example, for performing lithotripsy, RF sources, ultrasound emitters, electromagnetic sources, laser sources, thermal sources, and the like.

In connection therewith, various systems, apparatuses, methods, or the like are described herein that, in some examples, can help ensure the expandable medical device is expanded to its functional size when implanted within the body of the subject.

A defective native heart valve may be replaced with a transcatheter prosthetic heart valve in a transcatheter heart valve replacement procedure. Typically, the prosthetic heart valve can be implanted within a native valve annulus of the defective native heart valve using a delivery apparatus (which is also referred to herein as a “prosthetic heart valve delivery apparatus” and/or a “balloon catheter”). Typically, the delivery apparatus can comprise a shaft and an inflatable balloon mounted to a distal end portion of the shaft. The prosthetic heart valve can be mounted around the balloon, which can inflate to radially expand the prosthetic heart valve at the native valve annulus.

However, in some examples, the prosthetic heart valve may not be able to sufficiently conform to the geometry of the native tissue (for example, to the leaflets and/or annulus of the native heart valve) and may undesirably shift around relative to the native tissue, which can lead to paravalvular leakage. Thus, a docking device may be implanted first at the native valve annulus and then the prosthetic heart valve can be implanted within the docking device to help anchor the prosthetic heart valve to the native tissue and provide a seal between the native tissue and the prosthetic heart valve.

In some examples, the docking device may prevent at least a portion of the balloon and/or prosthetic heart valve from fully expanding to a functional size within the native valve annulus. For example, the docking device may constrain the balloon such that an intermediate portion of the balloon—and a corresponding intermediate portion of the prosthetic heart valve mounted around the balloon—can only partially expand in a radial direction. If the intermediate portion of the prosthetic heart valve is under-expanded to less than its functional size, the intermediate portion can form a valve “waist” having a smaller radial diameter than an inflow end portion and/or an outflow end portion of the prosthetic valve.

The inventors have discovered that fully expanding the intermediate portion of the prosthetic heart valve to its functional size, thereby eliminating or minimizing the valve waist, can beneficially further ensure that leaflets of the prosthetic heart valve exhibit normal function when the prosthetic heart valve is implanted within the native heart annulus. Furthermore, the inventors have discovered that eliminating the valve waist can further reduce the likelihood of left ventricular outflow tract obstruction (LVOTO). Finally, the inventors have discovered that eliminating the valve waist can result in less flaring of the inflow end portion and/or the outflow end portion of the prosthetic heart valve, which can beneficially further reduce any potential disturbance of nearby anatomical features and can result in better seating of the prosthetic heart valve within the native valve annulus. Thus, the inventors have discovered a need for ensuring that the intermediate portion of the prosthetic heart valve is expanded to its functional size during the transcatheter heart valve replacement procedure.

Thus, to address one or more problems of known balloon catheters, it would be desirable to design a balloon catheter to better expand a prosthetic heart valve within a docking device. For example, it would be desirable to design a balloon of a balloon catheter to exert more inflation pressure on an intermediate portion of the prosthetic heart valve than on an end portion of the prosthetic heart valve in order to more fully expand the intermediate portion of the prosthetic heart valve.

Described herein are some exemplary relative geometries of inflatable balloons that can, in some examples, help ensure that the inflatable balloons expand some examples of prosthetic heart valves (for example, prosthetic heart valve 450) in a way that beneficially results in a reduced likelihood of valve waist formation. For example, the disclosed balloons can be configured to exert a greater inflation pressure at intermediate portions of the exemplary heart valves than at proximal or distal end portions of the exemplary heart valves. This can help the intermediate portions of the exemplary heart valves better expand to a functional size, for example, when the intermediate portions are constrained in the radial direction by docking devices. However, it should be understood that the disclosed geometries may be specific to particular exemplary combinations of inflatable balloons, prosthetic heart valves, delivery apparatuses, docking devices, and/or procedures and that different balloon geometries may be more effective at reducing the likelihood of valve waist formation of different examples of prosthetic heart valves. Furthermore, it should be understood that any of the balloons disclosed herein can have any geometry.

### Examples of the Disclosed Technology

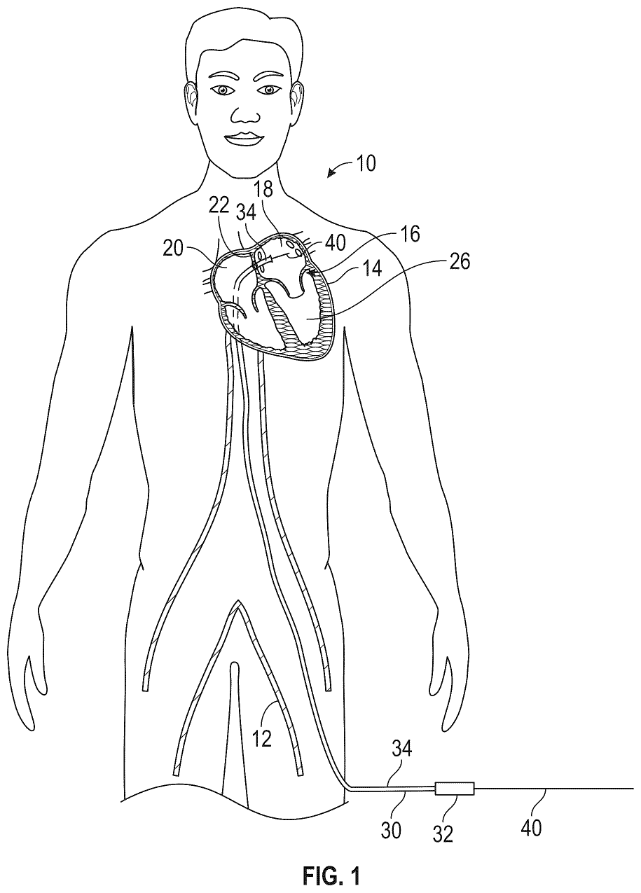

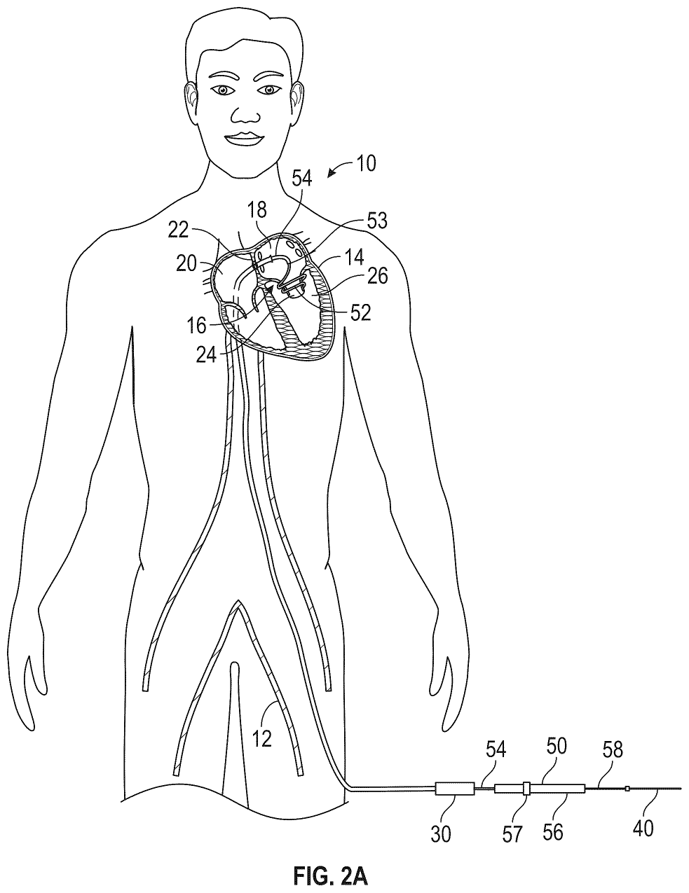

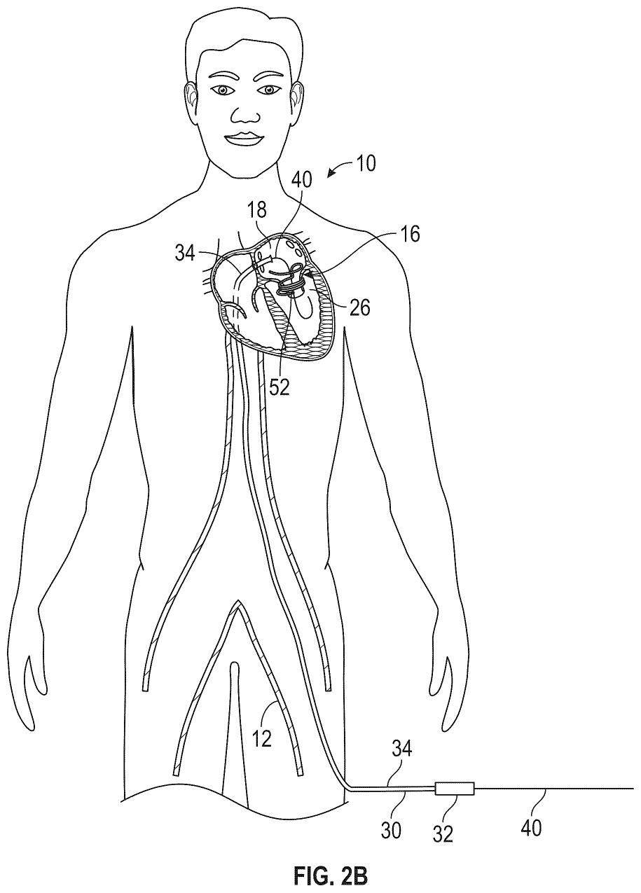

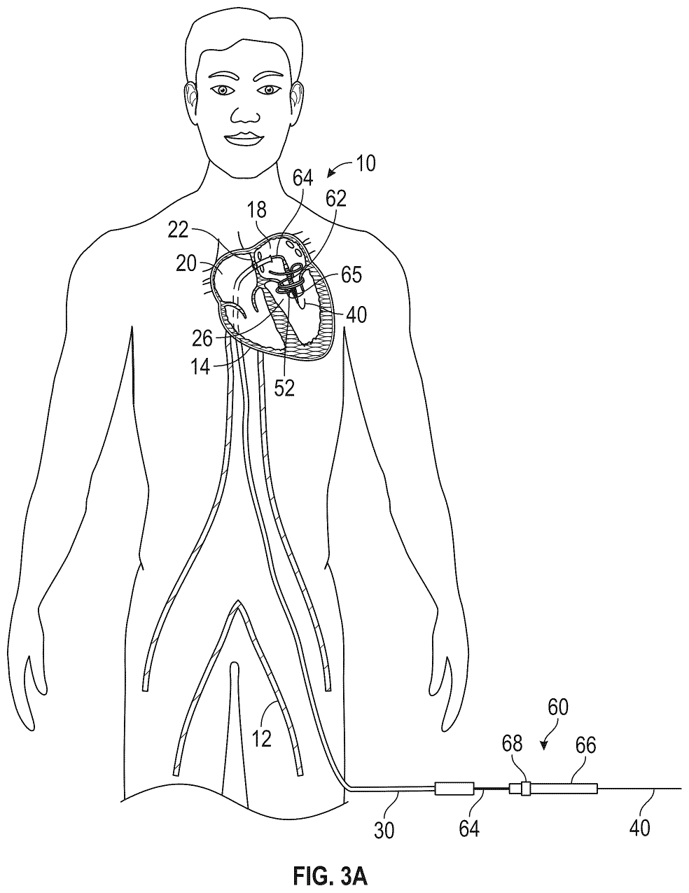

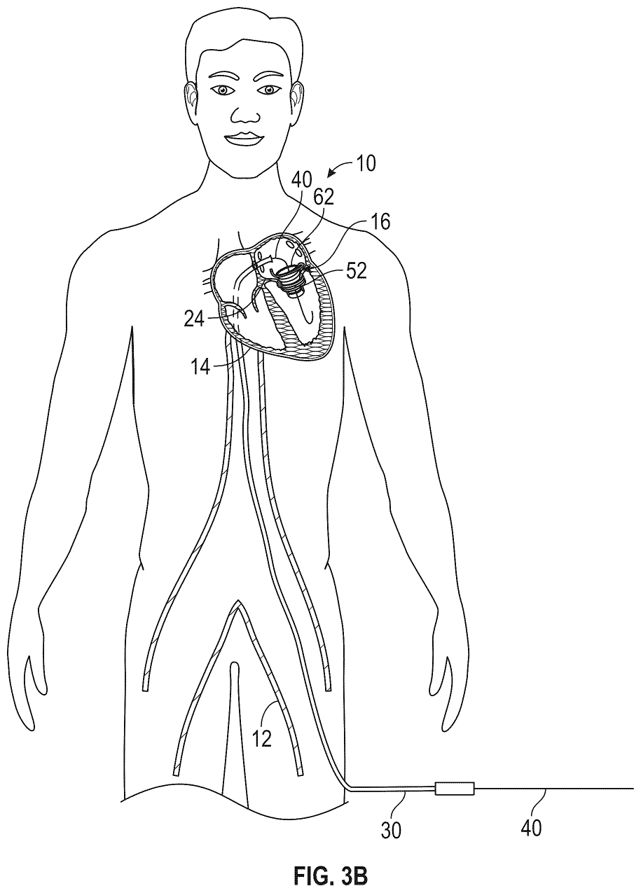

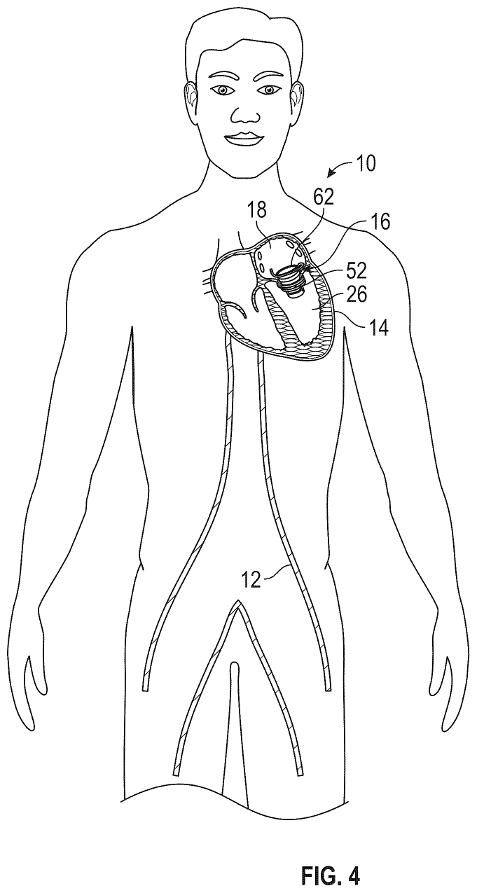

FIGS. 1-4 depict an example of a transcatheter heart valve replacement procedure (for example, a mitral valve replacement procedure) which utilizes a docking device 52 and a prosthetic heart valve 62, according to one example. During the procedure, a user first creates a pathway to a patient's native heart valve using a guide catheter 30 (FIG. 1). The user then delivers and implants the docking device 52 at the patient's native heart valve using a docking device delivery apparatus 50 (FIG. 2A) and then removes the docking device delivery apparatus 50 from the patient 10 after implanting the docking device 52 (FIG. 2B). The user then implants the prosthetic heart valve 62 within the implanted docking device 52 using a prosthetic valve delivery apparatus 60 (FIG. 3A). Thereafter, the user removes the prosthetic valve delivery apparatus 60 from the patient 10 (FIG. 3B), as well as the guide catheter 30 (FIG. 4).

FIG. 1 depicts a stage in a mitral valve replacement procedure, according to one example, where the guide catheter 30 and a guidewire 40 are inserted into a blood vessel 12 of a patient 10 and navigated through the blood vessel 12, into a heart 14 of the patient 10, and toward the native mitral valve 16. Together, the guide catheter 30 and the guidewire 40 can provide a path for the docking device delivery apparatus 50 and the prosthetic valve delivery apparatus 60 to be navigated through and along, to the implantation site (for example, the native mitral valve 16 or native mitral valve annulus). As shown, the heart 14 is illustrated schematically. For example, the anterior leaflet and chordae of the native mitral valve 16 are omitted for illustration purposes, such that only a portion of the posterior leaflet of the native mitral valve 16 is illustrated.

Initially, the user may first make an incision in the patient's body to access the blood vessel 12. For example, in the example illustrated in FIG. 1, the user may make an incision in the patient's groin to access a femoral vein. Thus, in such examples, the blood vessel 12 may be a femoral vein.

After making the incision at the blood vessel 12, the user may insert the guide catheter 30, the guidewire 40, and/or additional devices (for example, an introducer device or transseptal puncture device) through the incision and into the blood vessel 12. The guide catheter 30 (which can also be referred to as an “introducer device,” “introducer,” or “guide sheath”) is configured to facilitate the percutaneous introduction of various implant delivery devices (for example, the docking device delivery apparatus 50 and the prosthetic valve delivery apparatus 60) into and through the blood vessel 12 and may extend through the blood vessel 12 and into the heart 14 but may stop short of the native mitral valve 16. The guide catheter 30 can comprise a handle 32 and a shaft 34 extending distally from the handle 32. The shaft 34 can extend through the blood vessel 12 and into the heart 14 while the handle 32 remains outside the body of the patient 10 and can be operated by the user in order to manipulate the shaft 34 (FIG. 1).

The guidewire 40 is configured to guide the delivery apparatuses (for example, the guide catheter 30, the docking device delivery apparatus 50, the prosthetic valve delivery apparatus 60, additional catheters, or the like) and their associated devices (for example, docking device, prosthetic heart valve, and the like) to the implantation site within the heart 14, and thus may extend all the way through the blood vessel 12 and into a left atrium 18 of the heart 14 (FIG. 1) and in some examples, through the native mitral valve 16 and into a left ventricle of the heart 14.

In some instances, a transseptal puncture device or catheter can be used to initially access the left atrium 18, prior to inserting the guidewire 40 and the guide catheter 30. For example, after making the incision to the blood vessel 12, the user may insert a transseptal puncture device through the incision and into the blood vessel 12. The user may guide the transseptal puncture device through the blood vessel 12 and into the heart 14 (for example, through the femoral vein and into the right atrium 20). The user can then make a small incision in an atrial septum 22 of the heart 14 to allow access to the left atrium 18 from the right atrium 20. The user can then insert and advance the guidewire 40 through the transseptal puncture device within the blood vessel 12 and through the incision in the atrial septum 22 into the left atrium 18. Once the guidewire 40 is positioned within the left atrium 18 and/or the left ventricle 26, the transseptal puncture device can be removed from the patient 10. The user can then insert the guide catheter 30 into the blood vessel 12 and advance the guide catheter 30 into the left atrium 18 over the guidewire 40 (FIG. 1).

In some instances, an introducer device can be inserted through a lumen of the guide catheter 30 prior to inserting the guide catheter 30 into the blood vessel 12. In some instances, the introducer device can include a tapered end that extends out a distal tip of the guide catheter 30 and that is configured to guide the guide catheter 30 into the left atrium 18 over the guidewire 40. Additionally, in some instances the introducer device can include a proximal end portion that extends out a proximal end of the guide catheter 30. Once the guide catheter 30 reaches the left atrium 18, the user can remove the introducer device from inside the guide catheter 30 and the patient 10. Thus, only the guide catheter 30 and the guidewire 40 remain inside the patient 10. The guide catheter 30 is then in position to receive an implant delivery apparatus and help guide it to the left atrium 18, as described further below.

FIG. 2A depicts another stage in the example mitral valve replacement procedure where a docking device 52 is being implanted at the native mitral valve 16 of the heart 14 of the patient 10 using a docking device delivery apparatus 50 (which may also be referred to as an “implant catheter” and/or a “docking device delivery device”).

In general, the docking device delivery apparatus 50 comprises a delivery shaft 54, a handle 56, and a pusher assembly 58. The delivery shaft 54 is configured to be advanced through the patient's vasculature (blood vessel 12) and to the implantation site (for example, the native mitral valve 16) by the user and may be configured to retain the docking device 52 in a distal end portion 53 of the delivery shaft 54. In some examples, the distal end portion 53 of the delivery shaft 54 retains the docking device 52 therein in a straightened delivery configuration.

The handle 56 of the docking device delivery apparatus 50 is configured to be gripped and/or otherwise held by the user, outside the body of the patient 10, to advance the delivery shaft 54 through the patient's vasculature (for example, the blood vessel 12).

In some examples, the handle 56 can comprise one or more articulation members 57 (or rotatable knobs) that are configured to aid in navigating the delivery shaft 54 through the blood vessel 12. For example, the one or more articulation members 57 can comprise one or more of knobs, buttons, wheels, and/or other types of physically adjustable control members that are configured to be adjusted by the user to flex, bend, twist, turn, and/or otherwise articulate a distal end portion 53 of the delivery shaft 54 to aid in navigating the delivery shaft 54 through the blood vessel 12 and within the heart 14.

The pusher assembly 58 can be configured to deploy and/or implant the docking device 52 at the implantation site (for example, the native mitral valve 16). For example, the pusher assembly 58 is configured to be adjusted by the user to push the docking device 52 out of the distal end portion 53 of the delivery shaft 54. A shaft of the pusher assembly 58 can extend through the delivery shaft 54 and can be disposed adjacent the docking device 52 within the delivery shaft 54. In some examples, the docking device 52 can be releasably coupled to the shaft of the pusher assembly 58 via a connection mechanism of the docking device delivery apparatus 50 such that the docking device 52 can be released after being deployed at the native mitral valve 16.

Further details of the docking device delivery apparatus and its variants are described in International Publication No. WO 2020/247907, which is incorporated by reference herein in its entirety.

Referring again to FIG. 2A, after the guide catheter 30 is positioned within the left atrium 18, the user may insert the docking device delivery apparatus 50 (for example, the delivery shaft 54) into the patient 10 by advancing the delivery shaft 54 of the docking device delivery apparatus 50 through the guide catheter 30 and over the guidewire 40. In some examples, the guidewire 40 can be at least partially retracted away from the left atrium 18 and into the guide catheter 30. The user may then continue to advance the delivery shaft 54 of the docking device delivery apparatus 50 through the blood vessel 12 along the guidewire 40 until the delivery shaft 54 reaches the left atrium 18, as illustrated in FIG. 2A. Specifically, the user may advance the delivery shaft 54 of the docking device delivery apparatus 50 by gripping and exerting a force on (for example, pushing) the handle 56 of the docking device delivery apparatus 50 toward the patient 10. While advancing the delivery shaft 54 through the blood vessel 12 and the heart 14, the user may adjust the one or more articulation members 57 of the handle 56 to navigate the various turns, corners, constrictions, and/or other obstacles in the blood vessel 12 and the heart 14.

Once the delivery shaft 54 reaches the left atrium 18 and extends out of a distal end of the guide catheter 30, the user can position the distal end portion 53 of the delivery shaft 54 at and/or near the posteromedial commissure of the native mitral valve 16 using the handle 56 (for example, the articulation members 57). The user may then push the docking device 52 out of the distal end portion 53 of the delivery shaft 54 with the shaft of the pusher assembly 58 to deploy and/or implant the docking device 52 within the annulus of the native mitral valve 16.

In some examples, the docking device 52 may be constructed from, formed of, and/or comprise a shape memory material, and as such, may return to its original, pre-formed shape when it exits the delivery shaft 54 and is no longer constrained by the delivery shaft 54. As one example, the docking device 52 may originally be formed as a coil, and thus may wrap around leaflets 24 of the native mitral valve 16 as it exits the delivery shaft 54 and returns to its original coiled configuration.

After pushing a ventricular portion of the docking device 52 (for example, the portion of the docking device 52 shown in FIG. 2A that is configured to be positioned within a left ventricle 26 and/or on the ventricular side of the native mitral valve 16), the user may then deploy the remaining portion of the docking device 52 (for example, an atrial portion of the docking device 52) from the delivery shaft 54 within the left atrium 18 by retracting the delivery shaft 54 away from the posteromedial commissure of the native mitral valve 16.

After deploying and implanting the docking device 52 at the native mitral valve 16, the user may disconnect the docking device delivery apparatus 50 from the docking device 52. Once the docking device 52 is disconnected from the docking device delivery apparatus 50, the user may retract the docking device delivery apparatus 50 out of the blood vessel 12 and away from the patient 10 so that the user can deliver and implant a prosthetic heart valve 62 within the implanted docking device 52 at the native mitral valve 16.

FIG. 2B depicts this stage in the mitral valve replacement procedure, where the docking device 52 has been fully deployed and implanted at the native mitral valve 16 and the docking device delivery apparatus 50 (including the delivery shaft 54) has been removed from the patient 10 such that only the guidewire 40 and the guide catheter 30 remain inside the patient 10. In some examples, after removing the docking device delivery apparatus, the guidewire 40 can be advanced out of the guide catheter 30, through the implanted docking device 52 at the native mitral valve 16, and into the left ventricle 26 (FIG. 2A). As such, the guidewire 40 can help to guide the prosthetic valve delivery apparatus 60 through the annulus of the native mitral valve 16 and at least partially into the left ventricle 26.

As illustrated in FIG. 2B, the docking device 52 can comprise a plurality of turns (or coils) that wrap around the leaflets 24 of the native mitral valve 16 (within the left ventricle 26). The implanted docking device 52 has a more cylindrical shape than the annulus of the native mitral valve 16, thereby providing a geometry that more closely matches the shape or profile of the prosthetic heart valve to be implanted. As a result, the docking device 52 can provide a tighter fit, and thus a better seal, between the prosthetic heart valve and the native mitral valve 16, as described further below.

FIG. 3A depicts another stage in the mitral valve replacement procedure where the user is delivering and/or implanting a prosthetic heart valve 62 (which can also be referred to herein as a “transcatheter heart valve” or “THV” for short, “replacement heart valve,” and/or “prosthetic mitral valve”) within the docking device 52 using a prosthetic valve delivery apparatus 60.

As shown in FIG. 3A, the prosthetic valve delivery apparatus 60 can comprise a delivery shaft 64 and a handle 66, the delivery shaft 64 extending distally from the handle 66. The delivery shaft 64 is configured to extend into the patient's vasculature to deliver, implant, expand, and/or otherwise deploy the prosthetic heart valve 62 within the docking device 52 at the native mitral valve 16. The handle 66 is configured to be gripped and/or otherwise held by the user to advance the delivery shaft 64 through the patient's vasculature.

In some examples, the handle 66 can comprise one or more articulation members 68 that are configured to aid in navigating the delivery shaft 64 through the blood vessel 12 and the heart 14. Specifically, the articulation member(s) 68 can comprise one or more of knobs, buttons, wheels, and/or other types of physically adjustable control members that are configured to be adjusted by the user to flex, bend, twist, turn, and/or otherwise articulate a distal end portion of the delivery shaft 64 to aid in navigating the delivery shaft 64 through the blood vessel 12 and into the left atrium 18 and left ventricle 26 of the heart 14.

In some examples, the prosthetic valve delivery apparatus 60 can include an expansion mechanism 65 that is configured to radially expand and deploy the prosthetic heart valve 62 at the implantation site. In some instances, as shown in FIG. 3A, the expansion mechanism 65 can comprise an inflatable balloon that is configured to be inflated to radially expand the prosthetic heart valve 62 within the docking device 52. The inflatable balloon can be coupled to the distal end portion of the delivery shaft 64.

In other examples, the prosthetic heart valve 62 can be self-expanding and can be configured to radially expand on its own upon removable of a sheath or capsule covering the radially compressed prosthetic heart valve 62 on the distal end portion of the delivery shaft 64. In still other examples, the prosthetic heart valve 62 can be mechanically expandable and the prosthetic valve delivery apparatus 60 can include one or more mechanical actuators (for example, the expansion mechanism) configured to radially expand the prosthetic heart valve 62.

As shown in FIG. 3A, the prosthetic heart valve 62 is mounted around the expansion mechanism 65 (the inflatable balloon) on the distal end portion of the delivery shaft 64, in a radially compressed configuration.

To navigate the distal end portion of the delivery shaft 64 to the implantation site, the user can insert the prosthetic valve delivery apparatus 60 (the delivery shaft 64) into the patient 10 through the guide catheter 30 and over the guidewire 40. The user can continue to advance the prosthetic valve delivery apparatus 60 along the guidewire 40 (through the blood vessel 12) until the distal end portion of the delivery shaft 64 reaches the native mitral valve 16, as illustrated in FIG. 3A. More specifically, the user can advance the delivery shaft 64 of the prosthetic valve delivery apparatus 60 by gripping and exerting a force on (for example, pushing) the handle 66. While advancing the delivery shaft 64 through the blood vessel 12 and the heart 14, the user can adjust the one or more articulation members 68 of the handle 66 to navigate the various turns, corners, constrictions, and/or other obstacles in the blood vessel 12 and heart 14.

The user can advance the delivery shaft 64 along the guidewire 40 until the radially compressed prosthetic heart valve 62 mounted around the distal end portion of the delivery shaft 64 is positioned within the docking device 52 and the native mitral valve 16. In some examples, as shown in FIG. 3A, a distal end of the delivery shaft 64 and a least a portion of the radially compressed prosthetic heart valve 62 can be positioned within the left ventricle 26.

Once the radially compressed prosthetic heart valve 62 is appropriately positioned within the docking device 52 (FIG. 3A), the user can manipulate one or more actuation mechanisms of the handle 66 of the prosthetic valve delivery apparatus 60 to actuate the expansion mechanism 65 (for example, inflate the inflatable balloon), thereby radially expanding the prosthetic heart valve 62 within the docking device 52.

FIG. 3B shows another stage in the mitral valve replacement procedure where the prosthetic heart valve 62 in its radially expanded configuration and implanted within the docking device 52 in the native mitral valve 16. As shown in FIG. 3B, the prosthetic heart valve 62 is received and retained within the docking device 52. Thus, the docking device 52 aids in anchoring the prosthetic heart valve 62 within the native mitral valve 16. The docking device 52 can enable better sealing between the prosthetic heart valve 62 and the leaflets 24 of the native mitral valve 16 to reduce paravalvular leakage around the prosthetic heart valve 62.

As also shown in FIG. 3B, after the prosthetic heart valve 62 has been fully deployed and implanted within the docking device 52 at the native mitral valve 16, the prosthetic valve delivery apparatus 60 (including the delivery shaft 64) is removed from the patient 10 such that only the guidewire 40 and the guide catheter 30 remain inside the patient 10.

FIG. 4 depicts another stage in the mitral valve replacement procedure, where the guidewire 40 and the guide catheter 30 have been removed from the patient 10.

Although FIGS. 1-4 specifically depict a mitral valve replacement procedure, it should be appreciated that the same and/or similar procedure may be utilized to replace other heart valves (for example, tricuspid, pulmonary, and/or aortic valves). Further, the same and/or similar delivery apparatuses (for example, docking device delivery apparatus 50, prosthetic valve delivery apparatus 60, guide catheter 30, and/or guidewire 40), docking devices (for example, docking device 52), replacement heart valves (for example, prosthetic heart valve 62), and/or components thereof may be utilized for replacing these other heart valves.

For example, when replacing a native tricuspid valve, the user may also access the right atrium 20 via a femoral vein but may not need to cross the atrial septum 22 into the left atrium 18. Instead, the user may leave the guidewire 40 in the right atrium 20 and perform the same and/or similar docking device implantation process at the tricuspid valve. Specifically, the user may push the docking device 52 out of the delivery shaft 54 around the ventricular side of the tricuspid valve leaflets, release the remaining portion of the docking device 52 from the delivery shaft 54 within the right atrium 20, and then remove the delivery shaft 54 of the docking device delivery apparatus 50 from the patient 10. The user may then advance the guidewire 40 through the tricuspid valve into the right ventricle and perform the same and/or similar prosthetic heart valve implantation process at the tricuspid valve, within the docking device 52. Specifically, the user may advance the delivery shaft 64 of the prosthetic valve delivery apparatus 60 through the patient's vasculature along the guidewire 40 until the prosthetic heart valve 62 is positioned/disposed within the docking device 52 and the tricuspid valve. The user may then expand the prosthetic heart valve 62 within the docking device 52 before removing the prosthetic valve delivery apparatus 60 from the patient 10. In another example, the user may perform the same and/or similar process to replace the aortic valve but may access the aortic valve from the outflow side of the aortic valve via a femoral artery.

Further, although FIGS. 1-4 depict a mitral valve replacement procedure that accesses the native mitral valve 16 from the left atrium 18 via the right atrium 20 and femoral vein, it should be appreciated that the native mitral valve 16 may alternatively be accessed from the left ventricle 26. For example, the user may access the native mitral valve 16 from the left ventricle 26 via the aortic valve by advancing one or more delivery apparatuses through an artery to the aortic valve, and then through the aortic valve into the left ventricle 26.

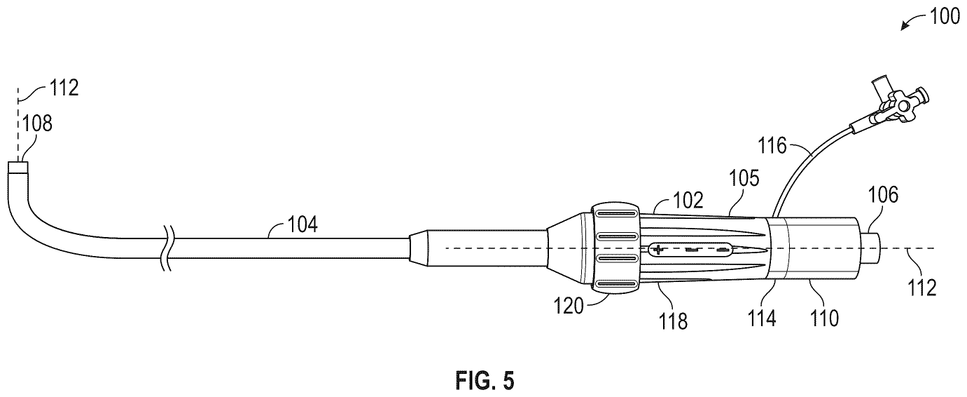

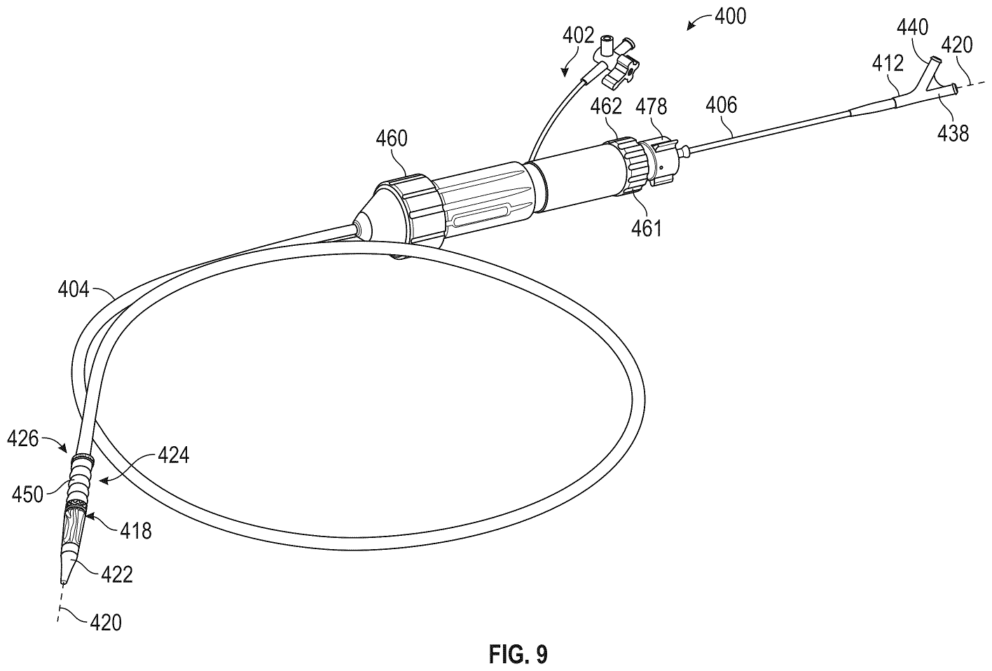

FIG. 5 illustrates an exemplary guide catheter, which is referred to below as a guide sheath 100 (and can also be referred to herein as a “delivery apparatus” or an “introducer device”). The guide sheath 100 can be configured to be inserted into a patient's vasculature and receive a balloon catheter or delivery apparatus therein in order to introduce the balloon catheter into the patient's vasculature and at least partially guide the balloon catheter therein to a target implantation site. For example, the guide sheath 100 can be used as the guide sheath 30 in a prosthetic valve implantation procedure, as described above with reference to FIGS. 1-4. An exemplary balloon catheter for a prosthetic medical device (referred to below as “delivery apparatus 400”) that can be received within the guide sheath 100 is shown in FIG. 9, as described further below. Though the guide sheath 100 is described herein as being used with the delivery apparatus 400, the guide sheath 100 can be configured to receive a variety of delivery apparatuses, balloon catheters, or implant catheters, such as alternate prosthetic heart valve delivery apparatuses, docking device delivery apparatuses, and/or delivery apparatuses for other prosthetic medical devices or medical therapies, such as stents.

The guide sheath 100 in the illustrated example comprises a handle 102, an elongated shaft 104 extending distally from the handle 102, and a central longitudinal axis 112. The shaft 104 has a main (or primary) lumen that is defined by an inner surface of a wall of the shaft 104. The main lumen is configured to receive a delivery apparatus therein (such as any of the prosthetic device delivery apparatuses or implant catheters described herein). In some examples, the shaft 104 can extend into the handle 102. Further, in some examples, the main lumen can extend through the handle 102 to an inlet port 106 disposed at a proximal end of the handle 102. Thus, in some examples, an inner surface of a wall of a portion of the handle (for example, at the proximal end) can further define the main lumen. Thus, the main lumen can extend from the inlet port 106 to a distal end 108 of the shaft 104.

The handle 102 can have an outer housing 105 and can further include a seal housing assembly 110 (which can also be referred to as a “seal stack”) which comprises one or more seals contained therein. The one or more seals of the seal housing assembly 110 can be configured to fluidly seal the main lumen of the guide sheath 100 from the external environment. For example, the one or more seals of the seal housing assembly 110 can be configured to prevent blood from a patient in which the guide sheath 100 is inserted from exiting the guide sheath 100 and prevent air from the environment from entering the guide sheath 100 (for example, through the inlet port 106). The one or more seals can include a variety of types of seals, such as a duckbill seal, a flapper seal, an umbrella valve, a cross-slit valve, a dome valve, or the like.

The handle 102 can, in some instances, include an adaptor spine 114 disposed adjacent and distal to the seal housing assembly 110. A flush port 116 can be connected to the outer housing 105 at the adaptor spine 114. A flush lumen of the adaptor spine 114 is connected to the flush port 116 and further connects to the main lumen. The flush port 116 can be configured to receive fluid through a lumen thereof. In this way, the flush port 116 can be fluidly coupled to the main lumen by the flush lumen.

The handle 102 can include a steering mechanism configured to adjust the curvature of the distal end portion of the shaft 104 (as such, the shaft 104 can be referred to as a steerable shaft). In the illustrated example, the handle 102 includes a main body portion 118 disposed adjacent and distal to the adaptor spine 114 and an adjustment member, such as the illustrated rotatable knob 120. The main body portion 118 can house internal flex mechanisms of the guide sheath 100 which are operatively coupled to the rotatable knob 120. In some examples, the flex mechanisms, and thus the knob 120, can be operatively coupled to the proximal end portion of a pull wire. The pull wire can extend distally from the handle 102 through the shaft 104 and have a distal end portion affixed to the shaft 104 at or near the distal end 108 of the shaft 104. Rotating the knob 120 can increase or decrease the tension in the pull wire, thereby adjusting the curvature of the distal end portion of the shaft 104. Further details on steering or flex mechanisms for a delivery apparatus can be found in U.S. Pat. No. 9,339,384, which is incorporated by reference herein.

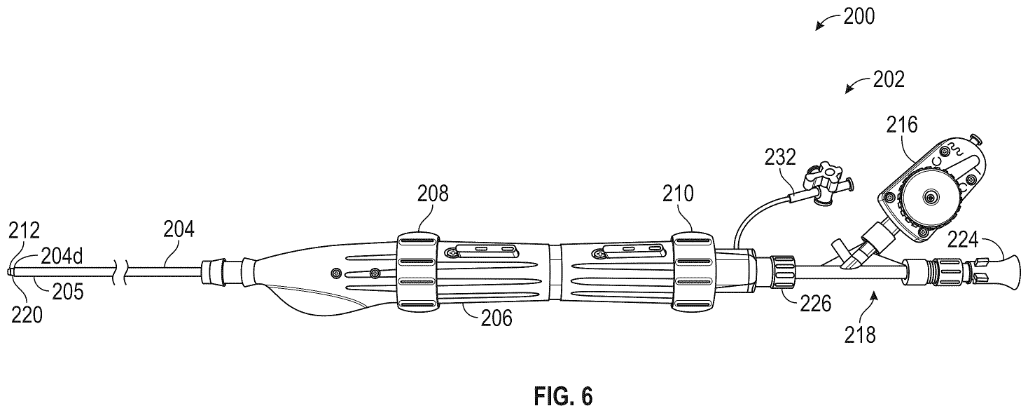

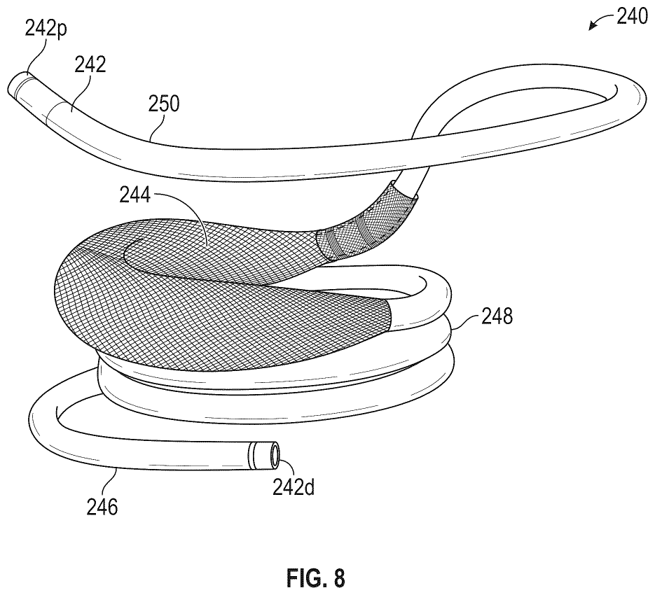

FIG. 6 illustrates a delivery apparatus 200 configured to implant a docking device, such as docking device 240 (FIG. 8) described below or other docking devices, to a target implantation site in a patient, according to one example. For example, the delivery apparatus 200 can be used as the docking device delivery apparatus 50 in a prosthetic valve implantation procedure, as described above with reference to FIG. 2A. The delivery apparatus 200 can also be referred to as a “dock delivery catheter” or “dock delivery system.”

As shown, the delivery apparatus 200 can include a handle assembly 202 and a delivery sheath 204 (also referred to as the “delivery shaft” or “outer shaft” or “outer sheath”) extending distally from the handle assembly 202. The handle assembly 202 can include a handle 206 including one or more knobs, buttons, wheels, and/or other means for controlling and/or actuating one or more components of the delivery apparatus 200. For example, in some examples, as shown in FIG. 6, the handle 206 can include knobs 208 and 210 which can be configured to steer or control flexing of the delivery apparatus 200 such as the delivery sheath 204 and/or a sleeve shaft 220 described below.

In certain examples, the delivery apparatus 200 can also include a pusher shaft 212 and a sleeve shaft 220, both of which can extend through an inner lumen of the delivery sheath 204 and have respective proximal end portions extending into the handle assembly 202.

As described below, a distal end portion (also referred to as “distal section”) of the sleeve shaft 220 can be configured to cover (for example, surround) the docking device 240 (see FIG. 8). For example, the docking device 240 can be retained inside the sleeve shaft 220, which is further retained by a distal end portion 205 of the delivery sheath 204, when navigating through a patient's vasculature.

Additionally, the distal end portion 205 of the delivery sheath 204 can be configured to be steerable. In one example, by rotating a knob (for example, one of knobs 208 or 210) on the handle 206, a curvature of the distal end portion 205 can be adjusted so that the distal end portion 205 of the delivery sheath 204 can be oriented in a desired angle. For example, to implant the docking device 240 at the native mitral valve location, the distal end portion 205 of the delivery sheath 204 can be steered in the left atrium so that at least a portion of the sleeve shaft 220 and the docking device 240 retained therein can extend through the native mitral valve annulus at a location adjacent the posteromedial commissure.

In certain examples, the pusher shaft 212 and the sleeve shaft 220 can be coaxial with one another, at least within the delivery sheath 204. In addition, the delivery sheath 204 can be configured to be axially movable relative to the sleeve shaft 220 and the pusher shaft 212. As described further below, a distal end of the pusher shaft 212 can be inserted into a lumen of the sleeve shaft 220 and press against the proximal end of the docking device 240 retained inside the sleeve shaft 220.

After reaching a target implantation site, the docking device 240 can be deployed from the delivery sheath 204 by manipulating the pusher shaft 212 and sleeve shaft 220 using a hub assembly 218, as described further below. For example, by pushing the pusher shaft 212 in the distal direction while holding the delivery sheath 204 in place or retracting the delivery sheath 204 in the proximal direction while holding the pusher shaft 212 in place, or pushing the pusher shaft 212 in the distal direction while simultaneously retracting the delivery sheath 204 in the proximal direction, the docking device 240 can be pushed out of a distal end 204d of the delivery sheath 204, thus changing from a delivery configuration to a deployed configuration (see FIG. 8). In certain examples, the pusher shaft 212 and the sleeve shaft 220 can be actuated independently of each other.

During delivery, the docking device 240 can be coupled to the delivery apparatus 200 via a release suture or other retrieval line comprising a string, yarn, or other material that can be configured to be tied around the docking device 240 and cut for removal that extends through the pusher shaft 212. In one specific example, the release suture can extend through the delivery apparatus 200, for example, through an inner lumen of the pusher shaft 212, to a suture lock assembly 216 of the delivery apparatus 200.

The handle assembly 202 can further include a hub assembly 218 to which the suture lock assembly 216 and a sleeve handle 224 are attached. The hub assembly 218 can be configured to independently control the pusher shaft 212 and the sleeve shaft 220 while the sleeve handle 224 can control an axial position of the sleeve shaft 220 relative to the pusher shaft 212. In this way, operation of the various components of the handle assembly 202 can actuate and control operation of the components arranged within the delivery sheath 204. In some examples, the hub assembly 218 can be coupled to the handle 206 via a connector 226.

The handle assembly 202 can further include one or more flush ports (for example, flush port 232 is shown in FIG. 6) to supply flush fluid to one or more lumens arranged within the delivery apparatus 200 (for example, annular lumens arranged between coaxial components of the delivery apparatus 200).

Further details on delivery apparatus/catheters/systems (including various examples of the handle assembly) that are configured to deliver a docking device to a target implantation site can be found in International Publication No. WO 2020/247907 and in U.S. Patent Publication Nos. 2018/0318079 and 2018/0263764, which are all incorporated by reference herein in their entireties.

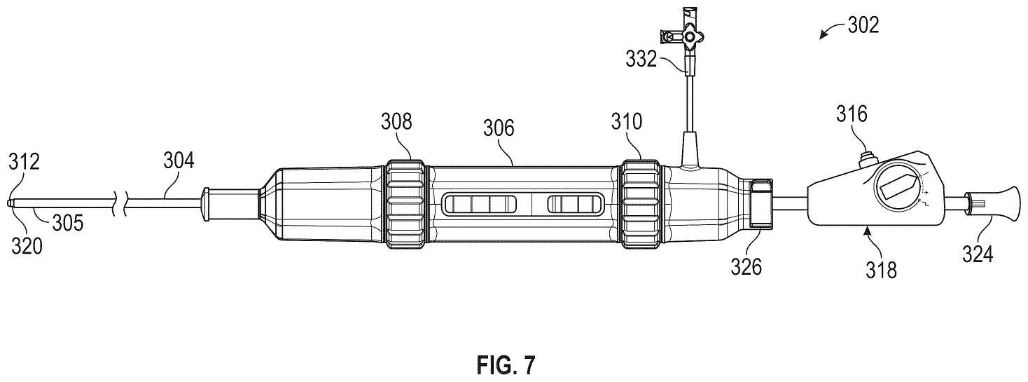

FIG. 7 illustrates a delivery apparatus 300 configured to deliver a docking device, according to an example. For convenience, similar reference numbers shown in FIG. 6 can describe components illustrated in FIG. 7. For example, handle assembly 302 can be similar to handle assembly 202, delivery sheath 304 can be similar to delivery sheath 204, distal end portion 305 can be similar to distal end portion 205, handle 306 can be similar to handle 206, knobs 308 and 310 can be similar to knobs 208 and 210, pusher shaft 312 can be similar to pusher shaft 212, suture lock assembly 316 can be similar to suture lock assembly 216, hub assembly 318 can be similar to hub assembly 218, sleeve shaft 320 can be similar to sleeve shaft 220, sleeve handle 324 can be similar to sleeve handle 224, connector 326 can be similar to connector 226, and flush port 332 can be similar to flush port 232.

FIG. 8 illustrates a docking device 240, according to one example. The docking device 240 can, for example, be used as the docking device 52 in a prosthetic valve implantation procedure, as described above with reference to FIGS. 1-4. As depicted in FIG. 8, the docking device in its deployed configuration can be configured to receive and secure a prosthetic valve therein, thereby securing the prosthetic valve at the native valve annulus.

The docking device 240 can comprise a coil 242 and a guard member 244 covering at least a portion of the coil 242. In some examples, the coil 242 can include a shape memory material (for example, nickel titanium alloy or “Nitinol”) such that the docking device 240 (and the coil 242) can move from a substantially straight configuration (or delivery configuration) when disposed within the delivery sheath 204 of the delivery apparatus 200 to a helical, deployed configuration after being removed from the delivery sheath 204.

The coil 242 has a proximal end 242p and a distal end 242d (which also respectively define the proximal and distal ends of the docking device 240). When being disposed within the delivery sheath 204 (for example, during delivery of the docking device 240 into the vasculature of a patient), a body of the coil 242 between the proximal end 242p and distal end 242d can form a generally straight delivery configuration (in other words, without any coiled or looped portions, but can be flexed or bent) so as to maintain a small radial profile when moving through a patient's vasculature. After being removed from the delivery sheath 204 and deployed at an implant position, the coil 242 can move from the delivery configuration to the helical deployed configuration and wrap around native tissue adjacent the implant position. For example, when implanting the docking device at the location of a native valve, the coil 242 can be configured to surround native leaflets of the native valve (and the chordae tendineae that connects native leaflets to adjacent papillary muscles, if present).

The docking device 240 can be releasably coupled to the delivery apparatus 200. For example, in certain examples, the docking device 240 can be coupled to a delivery apparatus (as described above) via a release suture that can be configured to be tied to the docking device 240 and cut for removal.

As shown in FIG. 8, the coil 242 in the deployed configuration can include a leading turn 246 (or “leading coil”), a central region 248, and a stabilization turn 250 (or “stabilization coil”) around a central longitudinal axis. The central region 248 can possess one or more helical turns having substantially equal inner diameters. The leading turn 246 can extend from a distal end of the central region 248 and has a diameter greater than the diameter of the central region 248, in the illustrated example. The stabilization turn 250 can extend from a proximal end of the central region 248 and has a diameter greater than the diameter of the central region 248, in the illustrated example. In some examples, the stabilization turn 250 can be omitted from the coil 242, for example, when a retention member (such as any retention member described herein) is used to stabilize the positioning of the docking device 240 relative to the native anatomy during an implant procedure. Alternatively, the stabilization turn 250 can have a diameter that is equal, approximately equal, or less than the diameter of the central region 248 (as opposed to larger), and/or the stabilization turn can comprise less of a full turn than depicted in FIG. 8.

Further details of the docking device and its variants are described in International Publication No. WO 2022/087336, which is incorporated by reference herein in its entirety, and International Publication No. WO 2020/247907.

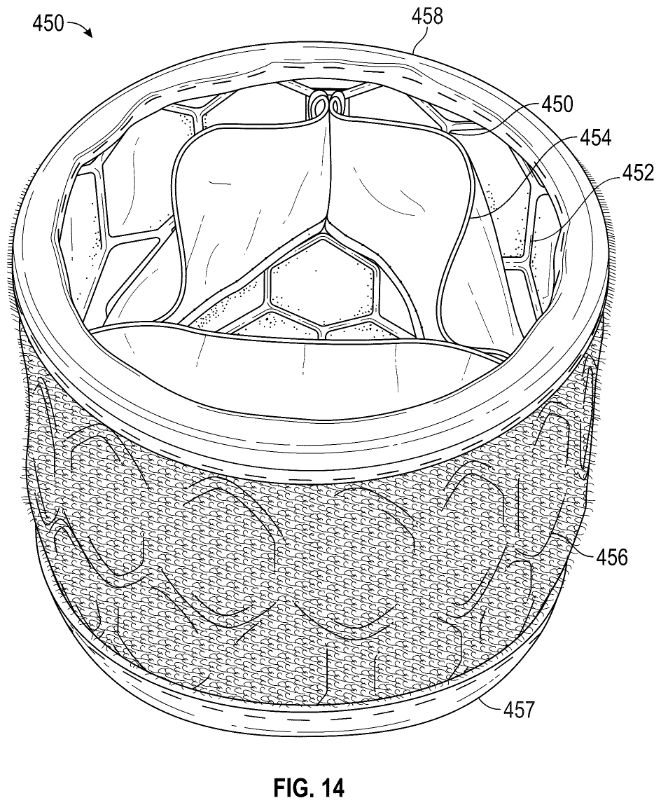

FIG. 9 illustrates a prosthetic heart valve delivery apparatus 400 (which can also be referred to here as an “implant catheter” and/or a “balloon catheter”) that can be used to implant an expandable prosthetic heart valve 450 (FIG. 14), according to one example. In some examples, the delivery apparatus 400 is specifically adapted for use in introducing a prosthetic heart valve into a heart. For example, the delivery apparatus 400 can be used as the prosthetic heart valve delivery apparatus 60 in a prosthetic valve implantation procedure, as described above with reference to FIG. 3A.

The delivery apparatus 400 in the illustrated example of FIG. 9 is a balloon catheter comprising a handle 402 and a steerable, outer shaft 404 extending distally from the handle 402. The delivery apparatus 400 can further comprise an intermediate shaft 406 (which also may be referred to as a balloon shaft) that extends proximally from the handle 402 and distally from the handle 402, the portion extending distally from the handle 402 also extending coaxially through the outer shaft 404. In some examples, the delivery apparatus 400 can further comprise an inner shaft extending distally from the handle 402 coaxially through the intermediate shaft 406 and the outer shaft 404 and proximally from the handle 402 coaxially through the intermediate shaft.

The outer shaft 404 and the intermediate shaft 406 can be configured to translate (e.g., move) longitudinally, along a central longitudinal axis 420 of the delivery apparatus 400, relative to one another to facilitate delivery and positioning of a prosthetic valve at an implantation site in a patient's body.

The intermediate shaft 406 can include a proximal end portion that extends proximally from a proximal end of the handle 402, to an adaptor 412. The adaptor 412 can include a first port 438 configured to receive a guidewire therethrough and a second port 440 configured to receive fluid (e.g., inflation fluid) from a fluid source. The second port 440 can be fluidly coupled to an inner lumen of the intermediate shaft 406.

In some examples, the intermediate shaft 406 can further include a distal end portion that extends distally beyond a distal end of the outer shaft 404 when a distal end of the outer shaft 404 is positioned away from an inflatable balloon 418 of the delivery apparatus 400. A distal end portion of the inner shaft can extend distally beyond the distal end portion of the intermediate shaft 406 toward or to a nose cone 422 at a distal end of the delivery apparatus 400.

In some examples, a distal end of the balloon 418 can be coupled to a distal end of the delivery apparatus 400, such as to the nose cone 422 (as shown in FIG. 9), or to an alternate component at the distal end of the delivery apparatus 400 (for example, a distal shoulder). An intermediate portion of the balloon 418 can overlay a valve mounting portion 424 of a distal end portion of the delivery apparatus 400 and a distal end portion of the balloon 418 can overly a distal shoulder of the delivery apparatus 400. As shown in FIG. 9, a prosthetic heart valve 450 can be mounted around the balloon 418, at the valve mounting portion 424 of the delivery apparatus 400, in a radially compressed state. The prosthetic heart valve 450 can be configured to be radially expanded by inflation of the balloon 418 at a native valve annulus, as described above with reference to FIG. 3A.

A balloon shoulder assembly of the delivery apparatus 400, which includes the distal shoulder, is configured to maintain the prosthetic heart valve 450 (or other medical device) at a fixed position on the balloon 418 during delivery through the patient's vasculature.

The outer shaft 404 can include a distal tip portion 426 mounted on its distal end. In some examples, the outer shaft 404 and the intermediate shaft 406 can be translated axially relative to one another to position the distal tip portion 426 adjacent a proximal end of the valve mounting portion 424, when the prosthetic valve 450 is mounted in the radially compressed state on the valve mounting portion 424 (as shown in FIG. 9) and during delivery of the prosthetic valve to the target implantation site. As such, the distal tip portion 426 can be configured to resist movement of the prosthetic valve 450 relative to the balloon 418 proximally, in the axial direction, relative to the balloon 418, when the distal tip portion 426 is arranged adjacent a proximal side of the valve mounting portion 424.

An annular space can be defined between an outer surface of the inner shaft and an inner surface of the intermediate shaft 406 and can be configured to receive fluid from a fluid source via the second port 440 of the adaptor 412. The annular space can be fluidly coupled to a fluid passageway formed between the outer surface of the distal end portion of the inner shaft and an inner surface of the balloon 418. As such, fluid from the fluid source can flow to the fluid passageway from the annular space to inflate the balloon 418 and radially expand and deploy the prosthetic valve 450.

An inner lumen of the inner shaft can be configured to receive a guidewire therethrough, for navigating the distal end portion of the delivery apparatus 400 to the target implantation site.

The handle 402 can include a steering mechanism configured to adjust the curvature of the distal end portion of the delivery apparatus 400. In the illustrated example, for example, the handle 402 includes an adjustment member, such as the illustrated rotatable knob 460, which in turn is operatively coupled to the proximal end portion of a pull wire. The pull wire can extend distally from the handle 402 through the outer shaft 404 and has a distal end portion affixed to the outer shaft 404 at or near the distal end of the outer shaft 404. Rotating the knob 460 can increase or decrease the tension in the pull wire, thereby adjusting the curvature of the distal end portion of the delivery apparatus 400. Further details on steering or flex mechanisms for the delivery apparatus can be found in U.S. Pat. No. 9,339,384.

The handle 402 can further include an adjustment mechanism 461 including an adjustment member, such as the illustrated rotatable knob 462, and an associated locking mechanism including another adjustment member, configured as a rotatable knob 478. The adjustment mechanism 461 is configured to adjust the axial position of the intermediate shaft 406 relative to the outer shaft 404 (for example, for fine positioning at the implantation site).

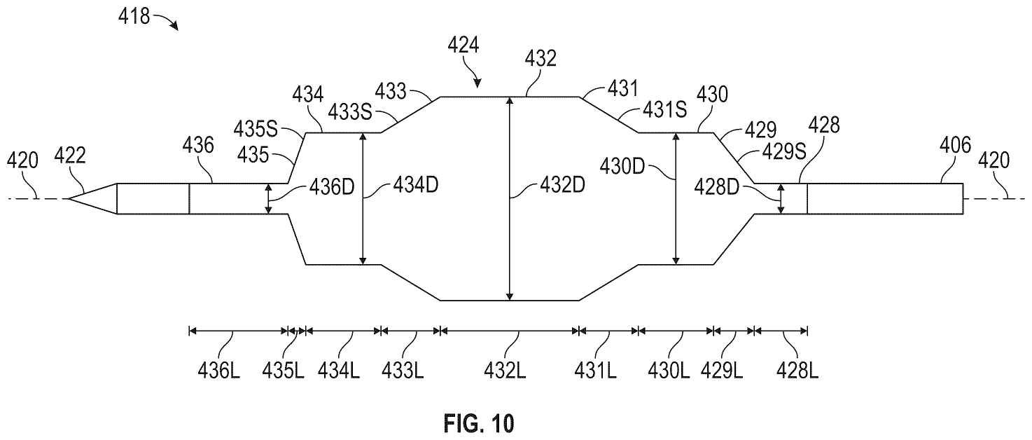

FIG. 10 is a side view of the distal end portion of the delivery apparatus 400 comprising the inflatable balloon 418, according to an example. As shown, the inflatable balloon 418 is in an inflated state. The inflatable balloon 418 (and the other balloons disclosed herein) comprise several portions along the axial length of the balloon that have different diameters. These differing diameters can either be tapered (see, for example, inflatable balloon 418) or step-wise (see, for example, inflatable balloon 518). Configuring a balloon in this manner can, for example, help ensure that the prosthetic implant (for example, a prosthetic heart valve) is cylindrical (or at least more cylindrical) upon expansion, particularly when the prosthetic implant is deployed within a docking or anchoring device.

In particular, the inflatable balloon 418 comprises a proximal neck portion 428 (which is also referred to herein as a “first neck portion”), a first proximal conical portion 429 (which is also referred to herein as a “first proximal frustoconical portion,” a “first conical portion,” and/or a “first frustoconical portion”), a proximal cylindrical portion 430 (which is also referred to herein as a “first cylindrical portion”), a second proximal conical portion (which is also referred to herein as a “second proximal frustoconical portion,” a “second conical portion,” and/or a “second frustoconical portion”), an intermediate portion 432, a second distal conical portion 433 (which is also referred to herein as a “second distal frustoconical portion,” a “third conical portion,” and/or a “third frustoconical portion”), a distal cylindrical portion 434 (which is also referred to herein as a “second cylindrical portion”), a first distal conical portion 435 (which is also referred to herein as a “first distal frustoconical portion,” a “fourth conical portion,” and/or a “fourth frustoconical portion”), and a distal neck portion 436.

The proximal neck portion 428 can be disposed at a proximal end of the inflatable balloon 418. The proximal neck portion 428 can have an axial length 428L and a radial diameter 428D. As shown, the proximal neck portion 428 of the inflatable balloon 418 can overlay a portion (for example, the distal end portion) of the intermediate shaft 406 of the delivery apparatus 400.

The first proximal conical portion 429 can be disposed between the proximal neck portion 428 and the proximal cylindrical portion 430. The first proximal conical portion 429 can have an axial length 429L. The first proximal conical portion 429 can taper from the proximal cylindrical portion 430 to the proximal neck portion 428. The taper of the first proximal conical portion 429 can define a slope 429S relative to the central longitudinal axis 420.

The proximal cylindrical portion 430 can be disposed between the first proximal conical portion 429 and the second proximal conical portion 431. The proximal cylindrical portion 430 can have an axial length 430L and a radial diameter 430D. The radial diameter 430D of the proximal cylindrical portion 430 can be greater than the radial diameter 428D of the proximal neck portion 428.

The second proximal conical portion 431 can be disposed between the proximal cylindrical portion 430 and the intermediate portion 432. The second proximal conical portion 431 can have an axial length 431L. The second proximal conical portion 431 can taper from the intermediate portion 432 to the proximal cylindrical portion 430. The taper of the second proximal conical portion 431 can define a slope 431S relative to the central longitudinal axis 420. As shown, the slope 431S can be less than the slope 429S of the first proximal conical portion 429. However, some examples of the slope 431S can be greater than or equal to the slope 429S of the first proximal conical portion 429.

The intermediate portion 432 can be disposed between the second proximal conical portion 431 and the second distal conical portion 433. The intermediate portion 432 can define an axial length 432L and a radial diameter 432D. The radial diameter 432D can be greater than each of the radial diameter 428D of the proximal neck portion 428 and the radial diameter 430D of the proximal cylindrical portion 430.

The second distal conical portion 433 can be disposed between the intermediate portion 432 and the distal cylindrical portion 434. The second distal conical portion 433 can have an axial length 433L. The second distal conical portion 433 can taper from the intermediate portion 432 to the proximal distal portion 434. The taper of the second distal conical portion 433 can define a slope 433S relative to the central longitudinal axis 420. As shown, the slope 433S of the second distal conical portion 433 can be greater than the slope 431S of the second proximal conical portion 431. However, some examples of the slope 435S of the first distal conical portion 435 can be less than or equal to the slope 429S of the first proximal conical portion.

The distal cylindrical portion 434 can be disposed between the second distal conical portion 433 and the first distal conical portion 435. The distal cylindrical portion 434 can have an axial length 434L and a radial diameter 434D. The radial diameter 434D of the distal cylindrical portion 430 can be less than the radial diameter 432D of the intermediate portion 432. As shown, the radial diameter 434D of the distal cylindrical portion 434 can be equal to the radial diameter 430D of the proximal cylindrical portion 430. However, some examples of the radial diameter 434D of the distal cylindrical portion 434 can be greater than or less than the radial diameter 430D of the proximal cylindrical portion 430.

The first distal conical portion 435 can be disposed between the distal cylindrical portion 434 and the distal neck portion 436. The first distal conical portion 435 can have an axial length 435L. As shown, the axial length 435L of the first distal conical portion 435 can be less than the axial length 429L of the first proximal conical portion. However, some examples of the axial length 435L of the first distal conical portion 435 can be greater than or equal to the axial length 429L of the first proximal conical portion.

The first distal conical portion 435 can taper from the distal cylindrical portion 434 to the distal neck portion 436. The taper of the first distal conical portion 435 can define a slope 435S relative to the central longitudinal axis 420. As shown, the slope 435S can be greater than the slope 433S of the second distal conical portion 433. However, some examples of the slope 435S can be less than or equal to the slope 433S of the second distal conical portion 433.

As shown, the slope 435S of the first distal conical portion 435 can be greater than the slope 429S of the first proximal conical portion 429. However, some examples of the slope 435S of the first distal conical portion 435 can be less than or equal to the slope 429S of the first proximal conical portion 429. As shown, the slope 435S of the first distal conical portion 435 can be greater than the slope 433S of the first distal conical portion 433. However, some examples of the slope 435S of the first distal conical portion 435 can be less than or equal to the slope 433S of the first distal conical portion 433.

The distal neck portion 436 can have an axial length 436L and a radial diameter 436D. In some examples, the distal neck portion 436 can overlay at least a portion of the nose cone 422 or the distal shoulder of the delivery apparatus 400.

As shown, the radial diameter 430D of the proximal cylindrical portion 430 and the radial diameter 434D of the distal cylindrical portion 434 can be equal. In some examples, the radial diameter 430D of the proximal cylindrical portion 430 can be different (for example, greater or less) than the radial diameter 434D of the distal cylindrical portion 434.

In some examples, forming the inflatable balloon 418 such that the radial diameter 432D is greater than each of the radial diameters 428D, 430D, 434D, and 436D can result in the inflatable balloon 418 applying more inflation pressure to an intermediate portion of the prosthetic heart valve 450 than to a distal and/or proximal end portion of the prosthetic heart valve 450. This configuration of the balloon 418 can beneficially help ensure that the intermediate portion of the prosthetic heart valve is expanded to its functional size upon deployment. By better ensuring that the intermediate portion of the prosthetic heart valve 450 is sufficiently expanded, this configuration of the inflatable balloon 418 can beneficially help reduce the likelihood that the intermediate portion of the prosthetic heart valve 450 forms a relatively narrow valve waist.

In some examples, the axial length 432L of the inflatable balloon 418 can be a certain proportion of the overall axial length of the inflatable balloon 418 to achieve certain design goals. In some examples, increasing the axial length 432L of the inflatable balloon 418 such that the axial length 432L is a greater proportion of the overall length of the inflatable balloon 418 can help provide a wider valve mounting portion 424 for easier alignment with the prosthetic heart valve 450 mounted therearound. In some examples, decreasing the axial length 432L of the inflatable balloon 418 such that the axial length 432L is a smaller proportion of the overall length of the inflatable balloon 418 can help concentrate inflation fluid and increase inflation pressure at the intermediate portion 432 to better expand the intermediate portion of the prosthetic heart valve 450 mounted therearound.

In some examples, the axial lengths of the various regions of the inflatable balloon 418 can be chosen such that the intermediate portion 432, which is configured to exert the greatest inflation pressure on the prosthetic heart valve 450 mounted therearound, better aligns with a portion of the prosthetic heart valve 450 most likely to form a valve waist. For example, if the portion of the prosthetic heart valve 450 most likely to form a valve waist is on a distal portion of the prosthetic heart valve, the sum of proximal axial lengths 428L, 429L, 430L, and 431L can be greater than the sum of distal axial lengths 433L, 434L, 435L, and 436L such that the intermediate portion 432 aligns with the relevant portion of the prosthetic heart valve 450. In some examples, the sum of proximal axial lengths 428L, 429L, 430L, and 431L can be less than the sum of distal axial lengths 433L, 434L, 435L, and 436L such that the intermediate portion 432 is more proximally disposed relative to the balloon 418. Thus, such examples of the balloon 418 can be axially asymmetrical.

The inflatable balloon 418 can be formed from any of various suitable thermoplastics and thermoset polymers. Examples of thermoplastics include polyolefins, polyamides, such as nylon 12, nylon 11, nylon 6/12, nylon 6, and nylon 66, polyesters, polyethers, polyurethanes, polyureas, polyvinyls, polyacrylics, fluoropolymers, copolymers and block copolymers thereof, such as block copolymers of polyether and polyamide, e.g., Pebax®; and mixtures thereof. Examples of thermosets include elastomers such as EPDM, epichlorohydrin, nitrile butadiene elastomers, silicones, etc. Thermosets, such as epoxies and isocyanates, can also be used. Biocompatible thermosets may also be used, and these include, for example, biodegradable polycaprolactone, poly(dimethylsiloxane) containing polyurethanes and ureas, and polysiloxanes. Any of the balloons disclosed herein can be made of one or more of any of these types of materials.

In some examples, the inflatable balloon 418 (or any other balloon disclosed herein) can be formed as a unitary component. In some examples, any of the various portions of the inflatable balloon 418 can be welded, adhered, fastened, or otherwise coupled together.

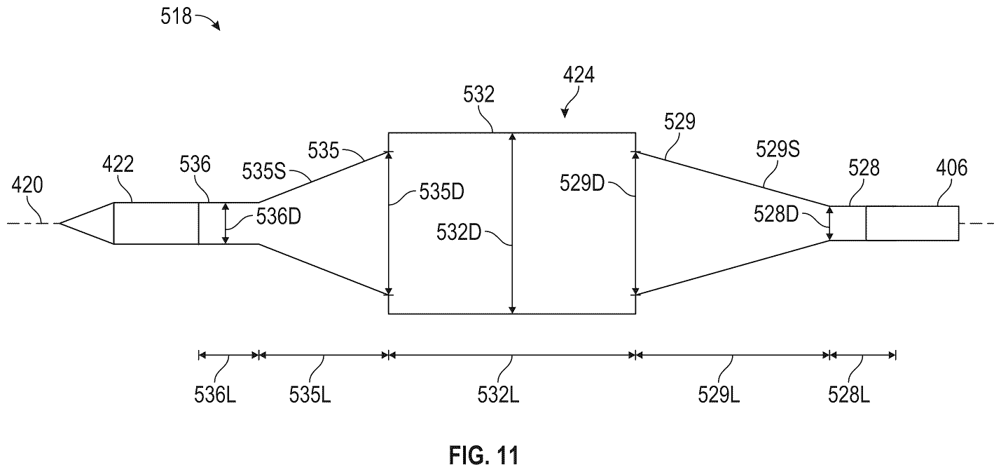

FIG. 11 is a side view of a distal end portion of the delivery apparatus 400 comprising an inflatable balloon 518, according to a second example. As shown, the inflatable balloon 518 comprises a proximal neck portion 528, a proximal conical portion 529 (which is also referred to herein as a “proximal frustoconical portion,” a “first conical portion,” and/or a “first frustoconical portion”) distally adjacent the proximal neck portion 528, an intermediate portion 532 distally adjacent the proximal conical portion 529, a distal conical portion 535 (which is also referred to herein as a “distal frustoconical portion,” a “second conical portion,” and/or a “second frustoconical portion”) distally adjacent the intermediate portion 532, and a distal neck portion 536 distally adjacent the distal conical portion 535. The proximal neck portion 528 can have an axial length 528L and a radial diameter 528D. The proximal conical portion 529 can have an axial length 529L and can taper to the proximal neck portion 528 at a slope 529S relative to the central longitudinal axis 420. The intermediate portion 532 can have an axial length 532L and a radial diameter 532D. The distal conical portion 535 can have an axial length 535L and can taper to the distal neck portion 536 at a slope 535S relative to the central longitudinal axis 420. The distal neck portion 536 can have an axial length 536L and a radial diameter 536D.

As shown, the axial length 532L of the intermediate portion 532 can be greater than the axial length 535L of the distal conical portion 535. However, some examples of the axial length 532L of the intermediate portion 532 can be less than or equal to the axial length 535L of the distal conical portion 535.

The radially widest portion of the proximal conical section 529 can define a radial diameter 529D. The radially widest portion of the distal conical section 535 can define a radial diameter 535D. As shown, the radial diameter 529D of the proximal conical section 529 can be equal to the radial diameter 535D of the distal conical section 535. However, some examples of the radial diameter 529D can be different (for example, greater or less) than the radial diameter 535D.

As shown, the radial diameter 532D of the intermediate portion 532 can be greater than each of a largest radial diameter 529D of the proximal conical portion 529 and a largest radial diameter 535D of the distal conical portion 535. This difference in radial diameters can result in the proximal and distal ends of the intermediate portion 532 forming “step-up” regions from the proximal conical portion 529 and the distal conical portion 535, respectively. The step-up regions, which extend in a substantially radial direction, can result in a more pronounced concentration of inflation pressure at the intermediate portion 532 of the balloon 518. However, some examples of the balloon 518, wherein the radial diameter 532D of the intermediate portion 532 is equal to the radial diameter 529D and/or the radial diameter 535D of the conical portions 529 and 535, do not form such step-up regions, resulting in improved manufacturability of the balloon 518.

The inflatable balloon 518 can be axially asymmetrical. For example, as shown, the axial length 528L of the proximal neck portion 528 can be less than the axial length 536L of the distal neck portion 536. Furthermore, the axial length 529L of the proximal conical portion 529 can be different than the axial length 535L of the distal conical portion 535. For example, as shown, the axial length 529L of the proximal conical portion 529 can be greater than the axial length 535L of the distal conical portion 535. In such examples, lengthening the axial length 529L of the proximal conical portion 529 relative to the axial length 535L of the distal conical portion 535 can result in the slope 535S of the distal conical portion 535 being greater or steeper than the slope 529S of the proximal conical portion 529. Designing the balloon 518 such that axial length 529L is greater than axial length 535L can better allow for the intermediate portion 532 to align with a portion of the prosthetic heart valve 450 (for example, the intermediate portion of the prosthetic heart valve 450) most likely to form a valve waist during deployment. However, some examples of the axial length 529L can be less than the axial length 535L to concentrate inflation pressures at other portions (for example, proximal portions) of the prosthetic heart valve 450.

Similar to axial length 432L illustrated in FIG. 10, the axial length 532L of the inflatable balloon 518 can be chosen relative to the overall axial length of the inflatable balloon 518 to achieve certain design goals. For example, the axial length 532L can be increased relative to the overall length of the balloon 518 to provide a wider valve mounting portion 424 for easier alignment with the prosthetic heart valve 450 mounted therearound, or can be decreased relative to the overall length of the balloon 518 to better target the concentration of inflation fluids in the intermediate portion 532, thereby resulting in greater inflation pressure exerted on portions of the prosthetic heart valve 450 most likely to form a valve waist.

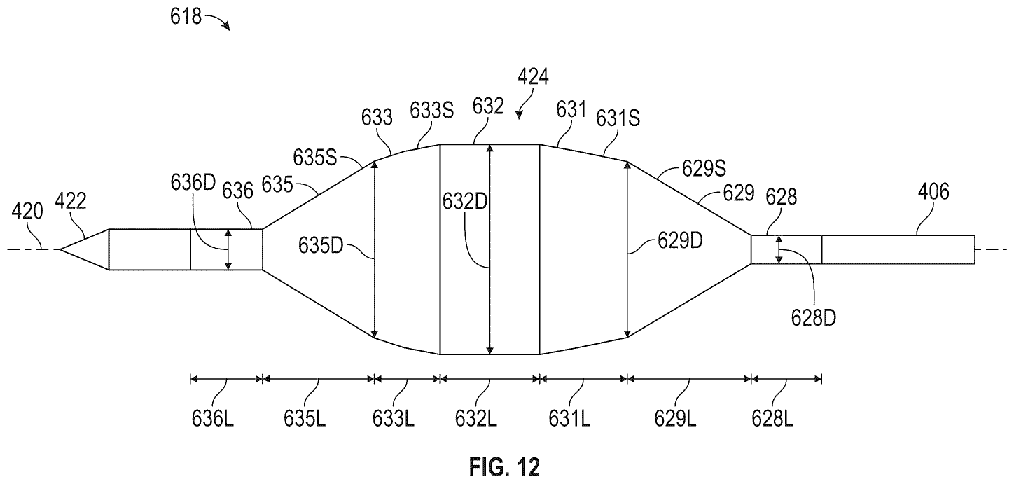

FIG. 12 is a side view of the distal end portion of the delivery apparatus 400 comprising an inflatable balloon 618, according to a third example. For convenience, similar reference numbers shown in FIG. 10 can describe components illustrated in FIG. 12. For example, proximal neck portion 428 can be similar to proximal neck portion 628, first proximal conical portion 429 can be similar to first proximal conical portion 629, second proximal conical portion 431 can be similar to second proximal conical portion 631, intermediate portion 432 can be similar to intermediate portion 632, second distal conical portion 433 can be similar to second distal conical portion 633, first distal conical portion 435 can be similar to first distal conical portion 635, and distal neck portion 436 can be similar to distal neck portion 636. One exemplary difference between the inflatable balloon 618 and the inflatable balloon 418 illustrated in FIG. 10 is that the inflatable balloon 618 lacks proximal and distal cylindrical portions (such as portions 430 and 434).

The proximal neck portion 628 (which is also referred to herein as a “first neck portion”) can define an axial length 628L and a radial diameter 628D. The proximal neck portion 628 can overlay a portion (for example, a portion of the intermediate shaft 406) of the delivery apparatus 400.

The first proximal conical portion 629 (which is also referred to herein as a “first proximal frustoconical portion,” a “first frustoconical portion,” and/or a “first conical portion”) can taper in the proximal direction from the second proximal conical portion 631 to the proximal neck portion 628. The first proximal conical portion 629 can define an axial length 629L, a slope 629S relative to the central longitudinal axis 420, and the widest portion of the first proximal conical portion 629 can define a radial diameter 629D.

As shown, the widest radial diameter 629D of the first proximal conical portion 629 can be greater than the axial length 629L of first proximal conical portion 629. For example, the widest radial diameter 629D of the first proximal conical portion 629 can be 33% to 53% greater than the axial length 629L of first proximal conical portion 629, such as 38% to 48% greater or 40% to 45% greater. However, some examples of the widest radial diameter 629D of the first proximal conical portion 629 can be less than or equal to the axial length 629L of first proximal conical portion 629.

The second proximal conical portion 631 (which is also referred to herein as a “second proximal frustoconical portion,” a “second frustoconical portion,” and/or a “second conical portion”) can taper in the proximal direction from the intermediate portion 432 to the first proximal conical portion 629. The second proximal conical portion 631 can define an axial length 631L and a slope 631S relative to the central longitudinal axis 420.

As shown, the axial length 629L of the first proximal conical portion 629 can be greater than the axial length 631L of the second proximal conical portion 631. For example, the axial length 629L of the first proximal conical portion 629 can be 33% to 53% greater than the axial length 631L of the second proximal conical portion 631, such as 38% to 48% greater or 40% to 45% greater. However, some examples of the axial length 629L of the first proximal conical portion 629 can be less than or equal to the axial length 631L of the second proximal conical portion 631.

As shown, the slope 629S of the first proximal conical portion 629 can be greater than the slope 631S of the second proximal conical portion 631. However, in some examples, the slope 629S of the first proximal conical portion 629 can be less than the slope 631S of the second proximal conical portion 631. In some examples, the slope 629S of the first proximal conical portion 629 can be equal to the slope 631S of the second proximal conical portion 631.

The intermediate portion 632, which can be disposed between the second proximal conical portion 631 and the second distal conical portion 633, can define an axial length 632L and a radial diameter 632D.

As shown, the axial length 629L of the first proximal conical portion 629 can be greater than the axial length 632L of the intermediate portion 632. For example, the axial length 629L of the first proximal conical portion 629 can be 15% to 35% greater than the axial length 632L of the intermediate portion 632, such as 20% to 30% greater or 22.5% to 27.5% greater. However, some examples of the axial length 629L of the first proximal conical portion 629 can be less than or equal to the axial length 632L of the intermediate portion 632.

The second distal conical portion 633 (which is also referred to herein as a “second distal frustoconical portion”) can taper from the intermediate portion 432 to the first distal conical portion 635. The second distal conical portion 633 can define an axial length 633L and a slope 633S relative to the central longitudinal axis 420.

As shown, the axial length 631L of the second proximal conical portion 631 can be greater than the axial length 633L of the second distal conical portion 633. For example, the axial length 631L of the second proximal conical portion 631 can be 22% to 42% greater than the axial length 633L of the second distal conical portion 633, such as 27% to 37% greater or 30% to 35% greater. However, some examples of the axial length 631L of the second proximal conical portion 631 can be less than or equal to the axial length 633L of the second distal conical portion 633.

As shown, the slope 633S of the second distal conical portion 633 can be greater than the slope 631S of the second proximal conical portion 631. However, some examples of the slope 633S of the second distal conical portion 633 can be less than or equal to the slope 631S of the second proximal conical portion 631.

The first distal conical portion 635 (which is also referred to herein as a “first distal frustoconical portion”) can taper from the second distal conical portion 633 to the distal neck portion 636. The first distal conical portion 635 can define an axial length 635L, a slope 635S relative to the central longitudinal axis 420, and the widest portion of the first distal conical portion 635 can define a radial diameter 635D.

As shown, the widest radial diameter 629D of the first proximal conical portion 629 can be equal to the widest radial diameter 635D of the first distal conical portion 635. However, some examples of the widest radial diameter 629D of the first proximal conical portion 629 can be greater than or less than to the widest radial diameter 635D of the first distal conical portion 635.

As shown, the axial length 629L of the first proximal conical portion 629 can be greater than the axial length 635L of the first distal conical portion 635. For example, the axial length 629L of the first proximal conical portion 629 can be 5% to 25% greater than the axial length 635L of the first distal conical portion 635, such as 5% to 15% greater or 7.5% to 12.5% greater. However, some examples of the axial length 629L of the first proximal conical portion 629 can be less than or equal to the axial length 635L of the first distal conical portion 635.

As shown, the axial length 635L of the first distal conical portion 635 can be greater than the axial length 632L of the intermediate portion 632. For example, the axial length 635L of the first distal conical portion 635 can be 5% to 25% greater than the axial length 632L of the intermediate portion 632, such as 7.5% to 17.5% greater or 10% to 15% greater. However, some examples of the axial length 635L of the first distal conical portion 635 can be less than or equal to the axial length 632L of the intermediate portion 632.

As shown, the widest radial diameter 635D of the first distal conical portion 635 can be greater than the axial length 635L of first distal conical portion 635. For example, the widest radial diameter 635D of the first distal conical portion 635 can be 50% to 70% greater than the axial length 635L of first distal conical portion 635, such as 55% to 65% greater or 57.5% to 62.5% greater. However, some examples of the widest radial diameter 635D of the first distal conical portion 635 can be less than or equal to the axial length 635L of first distal conical portion 635.

As shown, the slope 629S of the first proximal conical portion 629 can be less than the slope 635S of the first distal conical portion 635. However, in some examples, the slope 629S of the first proximal conical portion 629 can be greater than or equal to the slope 635S of the first distal conical portion 635.

As shown, the slope 635S of the first distal conical portion 635 can be greater than the slope 633S of the second distal conical portion 633. However, in some examples, the slope 635S of the first distal conical portion 635 can be less than or equal to the slope 633S of the second distal conical portion 633.

As shown, the axial length 635L of the first distal conical portion 635 can be greater than the axial length 633L of the second distal conical portion 633. For example, the axial length 635L of the first distal conical portion 635 can be 60% to 80% greater than the axial length 633L of the second distal conical portion 633, such as 65% to 75% greater or 67.5% to 72.5% greater. However, some examples of the axial length 635L of the first distal conical portion 635 can be less than or equal to the axial length 633L of the second distal conical portion 633.

The distal neck portion 636 (which is also referred to herein as a “second neck portion”) can define an axial length 636L and a radial diameter 636D. The distal neck portion 636 can overlay a portion (for example, the nose cone 422 or the distal shoulder) of the delivery apparatus 400.

As shown, the radial diameter 628D of the proximal neck portion 628 can be different than the radial diameter 636D of the distal neck portion 636. For example, the radial diameter 636D of the distal neck portion 636 can be 33% to 53% greater than the radial diameter 628D of the proximal neck portion 628, such as 38% to 48% greater or 40% to 45% greater. In some examples, the radial diameter 628D of the proximal neck portion 628 can be equal to the radial diameter 636D of the distal neck portion 636.

In some examples, the dimensions of the inflatable balloon 618 can be selected such that the inflatable balloon 618 applies a greater amount of inflation area to a particular portion of the prosthetic heart valve 450 mounted around the valve mounting portion 424. For example, as shown, the radial diameter 632D of the intermediate portion 632 can be greater than each of the widest radial diameter 629D of the first proximal conical portion 629 and the widest radial diameter 635D of the first distal conical portion 635. This balloon geometry can result in the intermediate portion 632 exerting a greater radial pressure upon an intermediate portion of the prosthetic heart valve 450 than upon a proximal and/or distal end portion of the prosthetic heart valve 450.

In some examples, the dimensions of the inflatable balloon 618 can be selected such that the intermediate portion 632 of the inflatable balloon 618 aligns with a portion of the prosthetic heart valve 450 most likely to form a valve waist when the prosthetic heart valve 450 is mounted around the valve mounting portion 424. For example, any of the axial length 629L of the first proximal conical portion 629, the axial length 631L of the second proximal conical portion 631, or the sum thereof can be greater than any of the axial length 633L of the second distal conical portion, the axial length 635L of the first distal conical portion 635, or the sum thereof such that the intermediate portion 632 of the balloon 618 aligns with a distal portion of the prosthetic heart valve 450 most likely to form a valve waist.

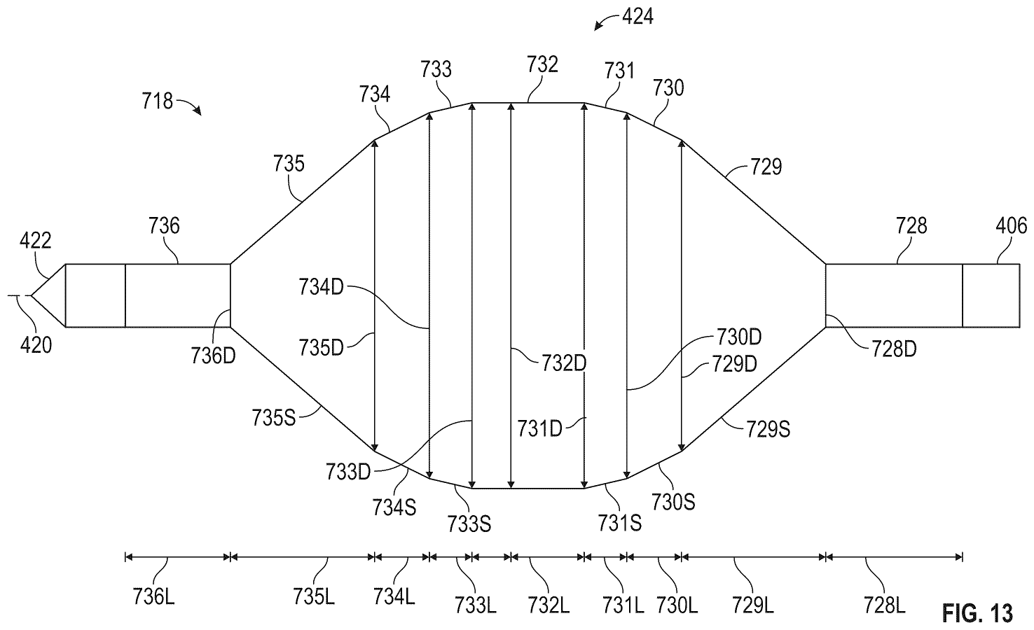

FIG. 13 is a side view of the distal end portion of the delivery apparatus 400 comprising an inflatable balloon 718, according to a fourth example. The balloon 718 comprises a first neck portion 728 (which is also referred to herein as a “proximal neck portion”), a first frustoconical portion 729 (which is also referred to herein as a “first proximal frustoconical portion,” a “first proximal conical portion,” and/or a “first conical portion”), a second frustoconical portion 730 (which is also referred to herein as a “second proximal frustoconical portion,” a “second proximal conical portion,” and/or a “second conical portion”), a third frustoconical portion 731 (which is also referred to herein as a “third proximal frustoconical portion,” a “third proximal conical portion,” and/or a “third conical portion”), a cylindrical intermediate portion 732, a fourth frustoconical portion 733 (which is also referred to herein as a “third distal frustoconical portion,” a “third distal conical portion,” and/or a “fourth conical portion”), a fifth frustoconical portion 734 (which is also referred to herein as a “second distal frustoconical portion,” a “second distal conical portion,” and/or a “fifth conical portion”), a sixth frustoconical portion 729 (which is also referred to herein as a “first distal frustoconical portion,” a “first distal conical portion,” and/or a “sixth conical portion”), and a second neck portion 736 (which is also referred to herein as a “distal neck portion”).

The first neck portion 728 can have an axial length 728L and a radial diameter 728D.

The first frustoconical portion 729 can be distally adjacent the first neck portion 728 and can taper from the second frustoconical portion 730 to the first neck portion 728. The first frustoconical portion 729 can have an axial length 729L and the radially widest portion of the first frustoconical portion 729 can define a radial diameter 729D. The first frustoconical portion 729 can taper in the proximal direction at a slope 729S relative to the central longitudinal axis 420.

The second frustoconical portion 730 can be distally adjacent the first frustoconical portion 729 and can taper from the third frustoconical portion 731 to the first frustoconical portion 729. The second frustoconical portion 730 can have an axial length 730L and the radially widest portion of the second frustoconical portion 730 can define a radial diameter 730D. The second frustoconical portion 730 can taper in the proximal direction at a slope 730S relative to the central longitudinal axis 420.

In some examples, the slope 730S of the second frustoconical portion 730 can be less than the slope 729S of the first frustoconical portion 729. However, some examples of the slope 730S of the second frustoconical portion 730 can be greater than or equal to the slope 729S of the first frustoconical portion 729.

The third frustoconical portion 731 can be distally adjacent the second frustoconical portion 730 and can taper from the intermediate portion 732 to the second frustoconical portion 730. The third frustoconical portion 731 can have an axial length 731L and the radially widest portion of the third frustoconical portion 731 can define a radial diameter 731D. The third frustoconical portion 731 can taper in the proximal direction at a slope 731S relative to the central longitudinal axis 420.

In some examples, the slope 731S of the third frustoconical portion 731 can be less than the slope 730S of the second frustoconical portion 730 and/or the slope 729S of the first frustoconical portion 729. However, some examples of the slope 731S of the third frustoconical portion 731 can be greater than or equal to the slope 730S of the second frustoconical portion 730 and/or the slope 729S of the first frustoconical portion 729.

As shown, the sum of the axial length 429L of the first frustoconical portion 429 and the axial length 430L of the second frustoconical portion can be greater than the axial length 431L of the third frustoconical portion 731. For example, the sum of the axial length 429L of the first frustoconical portion 429 and the axial length 430L of the second frustoconical portion can be 480% to 500% greater than the axial length 431L of the third frustoconical portion 731, such as 487% to 497% greater or 490% to 495% greater. However, some examples of the sum of the axial length 429L of the first frustoconical portion 429 and the axial length 430L of the second frustoconical portion can be less than or equal to the axial length 431L of the third frustoconical portion 731.

The intermediate portion 732 can have a radial diameter 732D and an axial length 732L.

In some examples, the sum of the axial length 729L of the first frustoconical portion 729 and the axial length 730L of the second frustoconical portion 730 can be greater than the radial diameter 732D of the intermediate portion 732. For example, the sum of the axial length 729L of the first frustoconical portion 729 and the axial length 730L of the second frustoconical portion 730 can be 0% to 20%, 0% to 10%, or 0% to 5% greater than the radial diameter 732D of the intermediate portion 732. However, some examples of the sum of the axial length 729L of the first frustoconical portion 729 and the axial length 730L of the second frustoconical portion 730 can be less than or equal to the radial diameter 732D of the intermediate portion 732.

As shown, the axial length 732L of the intermediate portion 732 can be greater than the axial length 731L of the third frustoconical portion 731. For example, the axial length 732L of the intermediate portion 732 can be 170% to 190% greater than the axial length 731L of the third frustoconical portion 731, such as 175% to 185% greater or 177.5% to 182.5% greater. However, some examples of the axial length 732L of the intermediate portion 732 can be less than or equal to the axial length 731L of the third frustoconical portion 731.

As shown, the radial diameter 732D of the intermediate portion 732 can be greater than the radial diameter 729D of the first frustoconical portion 729. For example, the radial diameter 732D of the intermediate portion 732 can be 5% to 30% greater than the radial diameter 729D of the first frustoconical portion 729, such as 10% to 25% greater or 15% to 20% greater. However, some examples of the radial diameter 732D of the intermediate portion 732 can be less than or equal to the radial diameter 729D of the first frustoconical portion 729.

As shown, the radial diameter 732D of the intermediate portion 732 can be greater than the axial length 732L of the intermediate portion 732. For example, the radial diameter 732D of the intermediate portion 732 can be 100% to 120% greater than the axial length 732L of the intermediate portion 732, such as 105% to 115% greater or 108% to 113% greater. However, some examples of the axial length 732L of the radial diameter 732D of the intermediate portion 732 can be less than or equal to the axial length 732L of the intermediate portion 732.

The fourth frustoconical portion 733 can be distally adjacent the intermediate portion 732 and can taper from the intermediate portion 732 to the fifth frustoconical portion 734. The fourth frustoconical portion 733 can have an axial length 733L and the radially widest portion of the fourth frustoconical portion 733 can define a radial diameter 733D. The fourth frustoconical portion 733 can taper in the distal direction at a slope 733S relative to the central longitudinal axis 420.

As shown, the axial length 731L of the third frustoconical portion 731 can be equal to the axial length 733L of the fourth frustoconical portion 733. However, some examples of the axial length 731L of the third frustoconical portion 731 can be greater than or less than the axial length 733L of the fourth frustoconical portion 733.

In some examples, the slope 733S of the fourth frustoconical portion 733 can be equal to the slope 731S of the third frustoconical portion 731. However, some examples of the slope 733S of the fourth frustoconical portion 733 can be greater than or less than the slope 731S of the third frustoconical portion 731.

As shown, the axial length 732L of the intermediate portion 732 can be greater than the axial length 733L of the fourth frustoconical portion 733. For example, the axial length 732L of the intermediate portion 732 can be 170% to 190% greater than the axial length 733L of the fourth frustoconical portion 733, such as 175% to 185% greater or 177.5% to 182.5% greater. However, some examples of the axial length 732L of the intermediate portion 732 can be less than or equal to the axial length 733L of the fourth frustoconical portion 733.

The fifth frustoconical portion 734 can be distally adjacent the fourth frustoconical portion 733 and can taper from the fourth frustoconical portion 733 to the sixth frustoconical portion 735. The fifth frustoconical portion 734 can have an axial length 734L and the radially widest portion of the fifth frustoconical portion 734 can define a radial diameter 734D. The fifth frustoconical portion 734 can taper in the distal direction at a slope 734S relative to the central longitudinal axis 420.

In some examples, the slope 734S of the fifth frustoconical section 734 can be greater than the slope 730S of the second frustoconical portion 730. However, some examples of the slope 734S of the fifth frustoconical section 734 can be less than or equal to the slope 730S of the second frustoconical portion 730.

In some examples, the axial length 734L of the fifth frustoconical section 734 can be less than the axial length 730L of the second frustoconical portion 730. However, some examples of the axial length 734L of the fifth frustoconical section 734 can be greater than or equal to the axial length 730L of the second frustoconical portion 730.

The sixth frustoconical portion 735 can be distally adjacent the fifth frustoconical portion 734 and can taper from the fifth frustoconical portion 734 to the second neck portion 736. The sixth frustoconical portion 735 can have an axial length 735L and the radially widest portion of the sixth frustoconical portion 735 can define a radial diameter 735D. The sixth frustoconical portion 735 can taper in the distal direction at a slope 735S relative to the central longitudinal axis 420.

As shown, the radial diameter 732D of the intermediate portion 732 can be greater than the radial diameter 735D of the sixth frustoconical portion 735. For example, the radial diameter 732D of the intermediate portion 732 can be 5% to 30% greater than the radial diameter 735D of the sixth frustoconical portion 735, such as 10% to 25% greater or 15% to 20% greater. However, some examples of the radial diameter 732D of the intermediate portion 732 can be less than or equal to the radial diameter 735D of the sixth frustoconical portion 735.

As shown, the axial length 729L of the first frustoconical portion 729 can be greater than the axial length 735L of the sixth frustoconical portion 735. For example, the axial length 729L of the first frustoconical portion 729 can be 10% to 30% greater than the axial length 735L of the sixth frustoconical portion 735, such as 10% to 25% greater or 15% to 20% greater. However, some examples of the first frustoconical portion 729 can be less than or equal to the axial length 735L of the sixth frustoconical portion 735.

In some examples, the radial diameter 732D of the intermediate portion 732 can be greater than the sum of the axial length 734L of the fifth frustoconical portion 734 and the axial length 735L of the sixth frustoconical portion 735. For example, the radial diameter 732D of the intermediate portion 732 can be 5% to 30% greater than the sum of the axial length 734L of the fifth frustoconical portion 734 and the axial length 735L of the sixth frustoconical portion 735, such as 10% to 25% greater or 15% to 20% greater. However, some examples of the radial diameter 732D of the intermediate portion 732 can be less than or equal to the sum of the axial length 734L of the fifth frustoconical portion 734 and the axial length 735L of the sixth frustoconical portion 735.

The second neck portion 736 can be distally adjacent the sixth frustoconical portion 735. The second neck portion 736 can have an axial length 736L and a radial diameter 736D.

Prosthetic valves disclosed herein (for example, prosthetic heart valve 450, prosthetic heart valve 62, etc.) can be radially compressible and expandable between a radially compressed state and a radially expanded state. Thus, the prosthetic valves can be crimped on or retained by an implant delivery apparatus (for example, delivery apparatus 400, delivery apparatus 60, etc.) in the radially compressed state during delivery, and then expanded to the radially expanded state once the prosthetic valve reaches the implantation site. It is understood that the prosthetic valves disclosed herein may be used with a variety of implant delivery apparatuses and can be implanted via various delivery procedures, examples of which will be discussed in more detail later.

FIG. 14 illustrates the prosthetic valve 450 in a radially expanded position. The prosthetic valve 450 can be used as the prosthetic heart valve 62 in a prosthetic valve implantation procedure, as described above with reference to FIGS. 1-4. Any of the prosthetic valves disclosed herein are adapted to be implanted in the native aortic annulus, although in other examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device (for example, docking device 240, docking device 52, etc.) that is implanted within a native heart valve or a vessel. For example, in one example, the disclosed prosthetic valves can be implanted within a docking device implanted within the pulmonary artery for replacing the function of a diseased pulmonary valve, such as disclosed in U.S. Publication No. 2017/0231756, which is incorporated by reference herein. In another example, the disclosed prosthetic valves can be implanted within a docking device implanted within or at the native mitral valve, such as disclosed in International Publication No. WO2020/247907. In another example, the disclosed prosthetic valves can be implanted within a docking device implanted within the superior or inferior vena cava for replacing the function of a diseased tricuspid valve, such as disclosed in U.S. Publication No. 2019/0000615, which is incorporated herein by reference.

The prosthetic valve 450 can be used as the prosthetic heart valve 62 in a prosthetic valve implantation procedure, as described above with reference to FIGS. 1-4. As shown in FIG. 14, the prosthetic valve 450 can include a frame 452 and a plurality of leaflets 454 situated at least partially within the frame 452. The prosthetic valve 450 can also include an outer covering 456 (which is also referred to herein as an “outer skirt”) situated about the frame 452. As shown in FIG. 14, the prosthetic valve 450 includes an inflow end 457 and an outflow end 458. The terms “inflow” and “outflow” are related to the normal direction of blood flow (for example, antegrade blood flow) through the prosthetic valve 450. For example, the leaflets 454 can allow blood flow through the valve 450 in a direction from the inflow end 457 to the outflow end 458 and prevent the reverse flow (for example, prevent flow in a direction from the outflow end 458 to the inflow end 457).

The frame 452 can be made of any of various suitable plastically-expandable materials (for example, stainless steel, etc.) or self-expanding materials (for example, Nitinol) as known in the art. When constructed of a plastically-expandable material, the frame 452 (and thus the valve 450) can be crimped to a radially compressed state on a delivery catheter and then expanded inside a patient by an inflatable balloon or equivalent expansion mechanism. When constructed of a self-expandable material, the frame 452 (and thus the valve 450) can be crimped to a radially compressed state and restrained in the compressed state by insertion into a sheath or equivalent mechanism of a delivery catheter. Once inside the body, the valve 450 can be advanced from the delivery sheath, which allows the valve 450 to expand to its functional size.

Suitable plastically-expandable materials that can be used to form the frames disclosed herein (for example, the frame 452) include, metal alloys, polymers, or combinations thereof. Example metal alloys can comprise one or more of the following: nickel, cobalt, chromium, molybdenum, titanium, or other biocompatible metal. In some examples, the frame 452 can comprise stainless steel. In some examples, the frame 452 can comprise cobalt-chromium. In some examples, the frame 452 can comprise nickel-cobalt-chromium. In some examples, the frame 452 comprises a nickel-cobalt-chromium-molybdenum alloy, such as MP35N™ (tradename of SPS Technologies), which is equivalent to UNS R30035 (covered by ASTM F562-2). MP35N™/UNS R30035 comprises 35% nickel, 35% cobalt, 20% chromium, and 10% molybdenum, by weight.

The outer skirt 456 can be wholly or partly formed of any suitable biological material, synthetic material (for example, any of various polymers), or combinations thereof. In some examples, the outer skirt 456 can comprise a fabric having interlaced yarns or fibers, such as in the form of a woven, braided, or knitted fabric. In some examples, the fabric can have a plush nap or pile. Exemplary fabrics having a plus nap or pile include velour, velvet, velveteen, corduroy, terrycloth, fleece, etc. In some examples, the outer skirt 456 can comprise a fabric without interlaced yarns or fibers or randomly interlaced yarns or fibers, such as felt or an electrospun fabric. Exemplary materials that can be used for forming such fabrics (with or without interlaced yarns or fibers) include, without limitation, polyethylene (PET), ultra-high molecular weight polyethylene (UHMWPE), polytetrafluoroethylene (PTFE), expanded polytetrafluoroethylene (ePTFE), polyamide etc. In some examples, the outer skirt 456 can comprise a non-textile or non-fabric material, such as a film made from any of a variety of polymeric materials, such as PTFE, PET, polypropylene, polyamide, polyetheretherketone (PEEK), polyurethane (such as thermoplastic polyurethane (TPU)), etc. In some examples, the outer skirt 456 can comprise a sponge material or foam, such as polyurethane foam. In some examples, the outer skirt 456 can comprise natural tissue, such as pericardium (for example, bovine pericardium, porcine pericardium, equine pericardium, or pericardium from other sources).

Further details of the prosthetic heart valve and its variants are described in U.S. Pat. No. 11,185,406, which is incorporated by reference herein in its entirety.

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (e.g., by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the prosthetic valves disclosed herein can be implanted using any of various delivery procedures and delivery devices known in the art.

### Sterilization

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### Simulation

The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A delivery apparatus for a prosthetic heart valve, comprising:
a shaft having a proximal end portion and a distal end portion; and
an inflatable balloon mounted on the distal end portion of the shaft, the balloon comprising:
   a first conical portion having a first axial length;
   a second conical portion axially disposed relative to the first conical portion, the second conical portion having a second axial length different than the first axial length, and
   a central longitudinal axis extending between the first conical portion and the second conical portion, wherein:
   the first conical portion has a first slope relative to the central longitudinal axis, and
   the second conical portion has a second slope relative to the central longitudinal axis that is different than the first slope.

Example 2. The delivery apparatus of any example herein, particularly example 1,wherein the inflatable balloon further comprises a cylindrical intermediate portion disposed between the first conical portion and the second conical portion.

Example 3. The delivery apparatus of any example herein, particularly example 2, wherein the cylindrical intermediate portion has an axial length, and the axial length of the cylindrical intermediate portion is greater than the second axial length of the second conical portion.

Example 4. The delivery apparatus of any example herein, particularly any one of examples 2-3, wherein the cylindrical intermediate portion comprises first and second ends, and wherein at least one of the first and second ends forms a step-up region extending substantially in a radial direction.

Example 5. The delivery apparatus of any example herein, particularly any one of examples 1-5, wherein the first axial length is greater than the second axial length.

Example 6. The delivery apparatus of any example herein, particularly any one of examples 1-6, wherein the first slope is less than the second slope.

Example 7. The delivery apparatus any example herein, particularly any one of examples 1-7, wherein the first conical portion and the second conical portion taper in opposite axial directions.

Example 8. A method comprising:
advancing a prosthetic heart valve mounted to the inflatable balloon of the delivery apparatus of any one of claims 1-7 to a native heart valve annulus of a patient;
positioning the prosthetic heart valve and the inflatable balloon within a docking device; and
inflating the balloon such that the prosthetic heart valve radially expands.

Example 9. A system comprising:
a docking device;
a prosthetic heart valve comprising a generally cylindrical stent frame with an axial opening, a proximal end portion, a distal end portion, and an intermediate portion between the proximal end portion and the distal end portion, the prosthetic heart valve configured to be positioned within the docking device; and
a balloon configured to extend through the axial opening of the stent frame, wherein the balloon comprises:
   a cylindrical intermediate portion;
   a proximal frustoconical portion tapering at a first slope relative to a central longitudinal axis of the balloon in a proximal direction from the cylindrical intermediate portion; and
   a distal frustoconical portion tapering at a second slope relative to the central longitudinal axis of the balloon in a distal direction from the cylindrical intermediate portion,
   wherein the intermediate portion, the proximal frustoconical portion, and the distal frustoconical portion are disposed along the central longitudinal axis;
   wherein the balloon, in an inflated state, is configured to exert a first pressure against the intermediate portion of the stent frame, and
   wherein the balloon, in the inflated state, is configured to expand and exert a second, lesser pressure against one of the proximal end portion and the distal end portion of the stent frame.

Example 10. The system of any example herein, particularly example 9, wherein the first slope is less than the second slope.

Example 11. The system of any example herein, particularly any one of examples 9-10, wherein the balloon further comprises a proximal cylindrical portion, and wherein the proximal frustoconical portion tapers from the cylindrical intermediate portion to the proximal cylindrical portion.

Example 12. The system of any example herein, particularly example 11, wherein the proximal frustoconical portion is a second proximal frustoconical portion, and wherein the balloon further comprises a first proximal frustoconical portion tapering from the proximal cylindrical portion.

Example 13. The system of any example herein, particularly example 12, wherein the first proximal frustoconical portion tapers from the proximal cylindrical portion at a third slope relative to the central longitudinal axis of the balloon, and wherein the third slope is greater than the first slope.

Example 14. The system of any example herein, particularly any one of examples 9-13, wherein the balloon further comprises a distal cylindrical portion, and wherein the distal frustoconical portion tapers from the cylindrical intermediate portion to the distal cylindrical portion.

Example 15. The system of any example herein, particularly example 14, wherein the distal frustoconical portion is a second distal frustoconical portion, and wherein the balloon further comprises a first distal frustoconical portion tapering from the distal cylindrical portion.

Example 16. The system of any example herein, particularly example 15, wherein the first distal frustoconical portion tapers from the distal cylindrical portion at a fourth slope relative to the central longitudinal axis of the balloon, and wherein the fourth slope is greater than the second slope.

Example 17. The system of any example herein, particularly any one of examples 9-16, wherein the proximal frustoconical portion comprises an axial length less than an axial length of the cylindrical intermediate portion.

Example 18. The system of any example herein, particularly any one of examples 9-17, wherein the proximal frustoconical portion comprises an axial length less than an axial length of the cylindrical intermediate portion.

Example 19. A balloon for a prosthetic implant delivery apparatus, comprising:
a central longitudinal axis;
a first neck portion;
a first conical portion tapering to the first neck portion at a first slope relative to the central longitudinal axis and in a first direction, the first conical portion having a first axial length;
a second conical portion tapering to the first conical portion at a second slope relative to the central longitudinal axis and in the first direction, the second conical portion having a second axial length;
an intermediate portion axially adjacent the second conical portion, the intermediate portion having an axial length and a radial diameter;
a third conical portion tapering from the intermediate portion at a third slope relative to the central longitudinal axis and in a second direction, the third conical portion having a third axial length;
a fourth conical portion tapering from the intermediate portion at a fourth slope relative to the central longitudinal axis and in the second direction, the fourth conical portion having a fourth axial length; and
a second neck portion.

Example 20. The balloon of any example herein, particularly example 19, wherein the widest portion of the first conical portion defines a radial diameter of the first conical portion, and wherein the radial diameter of the first conical portion is 33% to 53% greater than the axial length of the first conical portion.

Example 21. The balloon of any example herein, particularly any one of examples 19-20, wherein the first slope is greater than the second slope.

Example 22. The balloon of any example herein, particularly any one of examples 19-21, wherein the first axial length is 15% to 35% greater than the axial length of the intermediate portion.

Example 23. The balloon of any example herein, particularly any one of examples 19-22, wherein the second axial length is 22% to 42% greater than the third axial length.

Example 24. The balloon of any example herein, particularly any one of examples 19-23, wherein the third slope is greater than the second slope.

Example 25. The balloon any example herein, particularly any one of examples 19-24, wherein the first axial length is 5% to 25% greater than the fourth axial length.

Example 26. The balloon of any example herein, particularly any one of examples 19-25, wherein the first axial length is 5% to 25% greater than the axial length of the intermediate portion.

Example 27. The balloon of any example herein, particularly any one of examples 19-23, wherein the fourth slope is greater than the third slope.

Example 28. A balloon for a prosthetic implant delivery apparatus, comprising:
a central longitudinal axis;
a first frustoconical portion disposed on the central longitudinal axis, the first frustoconical portion having a first axial length and a first slope relative to the central longitudinal axis;
a second frustoconical portion axially adjacent the first frustoconical portion and tapering to the first frustoconical portion, the second frustoconical portion having a second axial length and a second slope relative to the central longitudinal axis;
a third frustoconical portion axially adjacent the second frustoconical portion and tapering to the second frustoconical portion, the third frustoconical portion having a third axial length and a third slope relative to the central longitudinal axis;
a cylindrical intermediate portion axially adjacent third frustoconical portion, the cylindrical intermediate portion having an axial length and a radial diameter;
a fourth frustoconical portion axially adjacent the cylindrical intermediate portion and tapering from the cylindrical intermediate portion, the fourth frustoconical portion having a fourth axial length and a fourth slope relative to the central longitudinal axis;
a fifth frustoconical portion axially adjacent the fourth frustoconical portion and tapering from the fourth frustoconical portion, the fifth frustoconical portion having a fifth axial length and a fifth slope relative to the central longitudinal axis; and
a sixth frustoconical portion axially adjacent the fifth frustoconical portion and tapering from the fifth frustoconical portion, the sixth frustoconical portion having a sixth axial length and a sixth slope relative to the central longitudinal axis.

Example 29. The balloon of any example herein, particularly example 28, wherein the second slope is greater than the first slope.

Example 30. The balloon of any example herein, particularly any one of examples 28-29, wherein the sum of the first axial length and the second axial length is 480% to 500% greater than the sum of the third axial length and the fourth axial length.

Example 31. The balloon of any example herein, particularly any one of examples 28-30, wherein the sum of the first and second axial lengths is 0% to 5% greater than the radial diameter of the cylindrical intermediate portion.

Example 32. The balloon of any example herein, particularly any one of examples 28-31, wherein the axial length of the intermediate portion 732 is 170% to 190% greater than the third axial length.

Example 33. The balloon of any example herein, particularly any one of examples 28-32, wherein the axial length of the intermediate portion 732 is 170% to 190% greater than the fourth axial length.

Example 34. The balloon of any example herein, particularly any one of examples 28-33, wherein the third axial length is equal to the fourth axial length.

Example 35. The balloon of any example herein, particularly any one of examples 28-34, wherein the radial diameter of the intermediate portion is 100% to 120% greater than the axial length of the intermediate portion.

Example 36. The balloon of any example herein, particularly any one of examples 28-35, wherein the fifth slope is greater than the fourth slope.

Example 37. The balloon of any example herein, particularly any one of examples 28-36, wherein the first axial length is 10% to 30% greater than the sixth axial length.

Example 38. The balloon of any example herein, particularly any one of examples 28-37, wherein the radial diameter of the intermediate portion is 5% to 30% greater than the sum of the fifth axial length and the sixth axial length.

Example 39. A method comprising:
implanting a docking device in a native annulus of a subject's heart;
using a delivery apparatus, advancing a prosthetic heart valve mounted to an inflatable balloon of the delivery apparatus to the native annulus;
positioning the prosthetic heart valve and the inflatable balloon within the docking device; and
inflating the balloon such that the balloon exerts a greater pressure on an intermediate portion of the prosthetic heart valve than one of a proximal end and a distal end of the prosthetic heart valve.

Example 40. A balloon of any example herein, particularly any one of examples 1-39, wherein the balloon is sterilized.

The features described herein with regard to any example can be combined with other features described in any one or more of the other examples, unless otherwise stated. For example, any one or more of the features of one balloon can be combined with any one or more features of another balloon. As another example, any one or more features of one delivery apparatus can be combined with any one or more features of another delivery apparatus.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

1. A delivery apparatus for a prosthetic heart valve, comprising:
a shaft having a proximal end portion and a distal end portion; and
an inflatable balloon mounted on the distal end portion of the shaft, the balloon comprising:
a first conical portion having a first axial length;
a second conical portion axially disposed relative to the first conical portion, the second conical portion having a second axial length different than the first axial length, and
a central longitudinal axis extending between the first conical portion and the second conical portion, wherein:
the first conical portion has a first slope relative to the central longitudinal axis, and
the second conical portion has a second slope relative to the central longitudinal axis that is different than the first slope.

2. The delivery apparatus of claim 1, wherein the inflatable balloon further comprises a cylindrical intermediate portion disposed between the first conical portion and the second conical portion.

3. The delivery apparatus of claim 2, wherein the cylindrical intermediate portion has an axial length, and the axial length of the cylindrical intermediate portion is greater than the second axial length of the second conical portion.

4. The delivery apparatus of claim 2, wherein the cylindrical intermediate portion comprises first and second ends, and wherein at least one of the first and second ends forms a step-up region extending substantially in a radial direction.

5. The delivery apparatus of claim 1, wherein the first axial length is greater than the second axial length.

6. The delivery apparatus of claim 1, wherein the first slope is less than the second slope.

7. A system comprising:
a docking device;
a prosthetic heart valve comprising a generally cylindrical stent frame with an axial opening, a proximal end portion, a distal end portion, and an intermediate portion between the proximal end portion and the distal end portion, the prosthetic heart valve configured to be positioned within the docking device; and
a balloon configured to extend through the axial opening of the stent frame, wherein the balloon comprises:
a cylindrical intermediate portion;
a proximal frustoconical portion tapering at a first slope relative to a central longitudinal axis of the balloon in a proximal direction from the cylindrical intermediate portion; and
a distal frustoconical portion tapering at a second slope relative to the central longitudinal axis of the balloon in a distal direction from the cylindrical intermediate portion,
wherein the intermediate portion, the proximal frustoconical portion, and the distal frustoconical portion are disposed along the central longitudinal axis;
wherein the balloon, in an inflated state, is configured to exert a first pressure against the intermediate portion of the stent frame, and
wherein the balloon, in the inflated state, is configured to expand and exert a second, lesser pressure against one of the proximal end portion and the distal end portion of the stent frame.

8. The system of claim 7, wherein the first slope is less than the second slope.

9. The system of claim 7, wherein the balloon further comprises a proximal cylindrical portion, and wherein the proximal frustoconical portion tapers from the cylindrical intermediate portion to the proximal cylindrical portion.

10. The system of claim 9, wherein the proximal frustoconical portion is a second proximal frustoconical portion, and wherein the balloon further comprises a first proximal frustoconical portion tapering from the proximal cylindrical portion.

11. The system of claim 10, wherein the first proximal frustoconical portion tapers from the proximal cylindrical portion at a third slope relative to the central longitudinal axis of the balloon, and wherein the third slope is greater than the first slope.

12. The system of claim 7, wherein the balloon further comprises a distal cylindrical portion, and wherein the distal frustoconical portion tapers from the cylindrical intermediate portion to the distal cylindrical portion.

13. The system of claim 12, wherein the distal frustoconical portion is a second distal frustoconical portion, and wherein the balloon further comprises a first distal frustoconical portion tapering from the distal cylindrical portion.

14. The system of claim 13, wherein the first distal frustoconical portion tapers from the distal cylindrical portion at a fourth slope relative to the central longitudinal axis of the balloon, and wherein the fourth slope is greater than the second slope.

15. A balloon for a prosthetic implant delivery apparatus, comprising:
a central longitudinal axis;
a first neck portion;
a first conical portion tapering to the first neck portion at a first slope relative to the central longitudinal axis and in a first direction, the first conical portion having a first axial length;
a second conical portion tapering to the first conical portion at a second slope relative to the central longitudinal axis and in the first direction, the second conical portion having a second axial length;
an intermediate portion axially adjacent the second conical portion, the intermediate portion having an axial length and a radial diameter;
a third conical portion tapering from the intermediate portion at a third slope relative to the central longitudinal axis and in a second direction, the third conical portion having a third axial length;
a fourth conical portion tapering from the intermediate portion at a fourth slope relative to the central longitudinal axis and in the second direction, the fourth conical portion having a fourth axial length; and
a second neck portion.

16. The balloon of claim 15, wherein the widest portion of the first conical portion defines a radial diameter of the first conical portion, and wherein the radial diameter of the first conical portion is 33% to 53% greater than the axial length of the first conical portion.

17. The balloon of claim 15, wherein the first slope is greater than the second slope.

18. The balloon of claim 15, wherein the first axial length is 15% to 35% greater than the axial length of the intermediate portion.

19. The balloon of claim 15, wherein the second axial length is 22% to 42% greater than the third axial length.

20. A balloon for a prosthetic implant delivery apparatus, comprising:
a central longitudinal axis;
a first frustoconical portion disposed on the central longitudinal axis, the first frustoconical portion having a first axial length and a first slope relative to the central longitudinal axis;
a second frustoconical portion axially adjacent the first frustoconical portion and tapering to the first frustoconical portion, the second frustoconical portion having a second axial length and a second slope relative to the central longitudinal axis;
a third frustoconical portion axially adjacent the second frustoconical portion and tapering to the second frustoconical portion, the third frustoconical portion having a third axial length and a third slope relative to the central longitudinal axis;
a cylindrical intermediate portion axially adjacent third frustoconical portion, the cylindrical intermediate portion having an axial length and a radial diameter;
a fourth frustoconical portion axially adjacent the cylindrical intermediate portion and tapering from the cylindrical intermediate portion, the fourth frustoconical portion having a fourth axial length and a fourth slope relative to the central longitudinal axis;
a fifth frustoconical portion axially adjacent the fourth frustoconical portion and tapering from the fourth frustoconical portion, the fifth frustoconical portion having a fifth axial length and a fifth slope relative to the central longitudinal axis; and
a sixth frustoconical portion axially adjacent the fifth frustoconical portion and tapering from the fifth frustoconical portion, the sixth frustoconical portion having a sixth axial length and a sixth slope relative to the central longitudinal axis.
